(19) United States
(12) Patent Application Publication
(10) Pub. No.: US 2023/0178177 A1
(43) Pub. Date: Jun. 8, 2023

(54) A SINGLE PATIENT CLASSIFIER FOR T1 HIGH GRADE BLADDER CANCER

(71) Applicants: Northwestern University, Evanston, IL (US); United States Government As Represented By The Department of Veterans Affairs, Washington, DC (US); Dxige Research, Inc., Courtenay (CA)

(72) Inventors: Joshua J. Meeks, Western Springs, IL (US); Andrew Gordon Robertson, Courtenay (CA); Aurélien de Reyniès, Paris (FR); Clarice S. Groeneveld, Paris (FR)

(21) Appl. No.: 18/003,911

(22) PCT Filed: Jun. 30, 2021
(86) PCT No.: PCT/US2021/070809

§ 371 Date: Dec. 29, 2022

Related U.S. Application Data
(60) Provisional application No. 63/046,467, filed on Jun. 30, 2020.

(51) Int. Cl. G16B 25/10; G16H 50/30

(52) U.S. Cl. CPC G16B 25/10; G16H 50/30

(57) ABSTRACT

Disclosed are methods and systems for diagnosing, staging, stratifying, prognosing, and/or treating bladder cancer. In particular, the invention relates to methods and systems for classifying Stage T1 bladder cancer. The disclosed methods may include generating an mRNA expression profile for a sample of Stage T1 bladder cancer and classifying the sample based on the expression profile.

### CROSS-REFERENCE TO RELATED PATENT APPLICATIONS

The present application claims the benefit of priority under 35 U.S.C. 119(e) to U.S. Provisional Application No. 63/046,467, filed on Jun. 30, 2020, the content of which is incorporated herein by reference in its entirety.

### STATEMENT REGARDING FEDERALLY SPONSORED RESEARCH OR DEVELOPMENT

This invention was made with government support under VHA BX003692-01 awarded by the Veterans Health Administration and W81XWH-18-0257 awarded by the Department of Defense. The government has certain rights in the invention.

### BACKGROUND

The invention relates to methods and systems for diagnosing, staging, stratifying, prognosing, and/or treating bladder cancer. In particular, the invention relates to methods and systems for classifying Stage T1 bladder cancer. The disclosed methods may include generating an mRNA expression profile for a sample of Stage T1 bladder cancer and classifying the sample based on the expression profile.

Stage T1 bladder cancers have the highest progression and recurrence rates of all non-muscle invasive bladder cancers (NMIBC). Most T1 cancers are treated with BCG, but many will progress or recur, and some T1 patients will die from bladder cancer. Particularly aggressive tumors could be treated by early cystectomy. To better understand the molecular heterogeneity of T1 cancers, the present inventors performed transcriptome profiling and unsupervised clustering, identifying five consensus subtypes of T1 tumors treated with reTUR, induction and maintenance BCG. The T1-LumGU subtype was associated with CIS (6/13, 46% of all CIS), had high E2F1 and EZH2 expression, and enriched E2F target and G2M checkpoint Hallmarks. T1-Inflam was inflamed and infiltrated with immune cells. While most T1 tumors were classified as luminal papillary, the T1-TLum subtype had the highest median Luminal Papillary score and FGFR3 expression, no recurrence events, and the fewest copy number gains. T1-Myc and T1-Early subtypes had the most recurrences (14/30 within 24 months), highest median MYC expression, and, when combined, had significantly worse recurrence-free survival than the other three subtypes. T1-Early had 5 (38%) recurrences within the first 6 months of BCG, and repressed gene sets for inflammation, and IFN-alpha and IFN-gamma Hallmarks. The inventors developed a single-patient T1 classifier and validated their subtype biology in a second cohort of T1 tumors.

### SUMMARY

Disclosed are methods and systems for diagnosing, staging, stratifying, prognosing, and/or treating bladder cancer. In particular, the invention relates to methods and systems for classifying Stage T1 bladder cancer. The disclosed methods may include generating an mRNA expression profile for a sample of Stage T1 bladder cancer and classifying the sample based on the expression profile.

The disclosed methods and systems may utilize or include one or more detecting steps and/or classifying steps that are performed on biological sample, such as a sample of bladder tissue having or at risk for developing bladder cancer. In some aspects, the methods and systems may utilize or include one or more of the following detecting steps: (a) detecting activation and/or repression in the sample of one or more regulons selected from E2F1, TP63, and ZNF385A; (b) detecting an increase in somatic copy number; (c) detecting expression in the sample of one or more genes selected from FOXM1, RXRA, MYC, E2F1, EZH2, FGFR3, STAT4, and NFATC2; (d) detecting tumor purity for the sample; (e) detecting an immune score and/or a stromal score for the sample; (f) detecting immune cells in the sample; (g) detecting inflammation in the sample; (h) detecting carcinoma in situ (CIS) in the sample, and/or detecting expression and/or repression of genes associated with CIS in the sample; (i) detecting expression of one or more genes selected from E2F target genes, G2M checkpoints genes, inflammatory response genes, IL2/STAT5 signaling genes, IFNG response genes, INFA response genes, and MYC target genes; thereby classifying the Stage T1 bladder cancer. Optionally, the methods and systems may utilize and include a classifying step utilizing known subtype classifiers. In some aspects, the classifying step may include (j) classifying the sample by one or more subtype classifiers selected from Lund, TCGA mRNA subtype classifier, consensusMIBC, and UROMOL class.

### BRIEF DESCRIPTION OF THE FIGURES

FIG. 1. Characterization of five gene expression subtypes T1 tumors based on transcriptome profiles and clinical variables. A) Top: A heatmap of subtype-specific differentially expressed genes (DEGs) for five unsupervised consensus expression clusters identified using the most-variant 2945 (i.e. 15%) protein-coding genes (see Supplemental Methods). Below the DEG heatmap is a heatmap showing activity status profiles for 33 regulons, with red, blue and grey respectively indicating activated, repressed, and undefined regulon activity status. Below this are covariate tracks for expression-based classifier subtyping of each T1 tumor, with consensusMIBC, TCGA, UROMOL and Lund subtypes shown. Below these are clinical and pathologic covariates. B) Kaplan-Meier plot for recurrence, censored at 24 mo, with a log-rank p-value, demonstrating increased recurrence of subtypes T1-Myc (S3, 24 months) and T1-Early (S5, 6 mo). C) Selected MSigDB Hallmark gene sets that were enriched in genes that were overexpressed (red disks) or underexpressed (blue) in a subtype. GSEA results are from CERNO tests [5]; disk diameter is proportional to AUC (i.e. effect size), and color opacity is proportional to −log 10(p_{Adj}). D) CERNO tests of 170 inflammation-related genes and CIS UP/DOWN genes, with dot size and color as described in (C). E) Distribution of LumP consensusMIBC classifier scores across T1 subtypes; T1-TLum had the highest median LumP score. F) Cytotoxic lymphocytes predicted by MCPcounter (FIG. 7); T1-Inflam had the most immune cells. G) Somatic copy number (CN) gains, expressed as a fraction of the total genome length with CN calls; T1-LumGU had the most CNAs and T1-TLum the fewest. H) Per-subtype expression distributions of select genes. For a comparison of FPKM and TPM expression distributions, see FIG. 5B. I) A Kaplan-Meier curve identified significantly worse recurrence at 24 months for the two highest-recurrence subtypes (T1-Myc and T1-Early, S3/5), compared to the three other subtypes (T1-LumGU/Inflam/Lum, S1/2/4). J) The regulon-based group consisting of subtypes T1-LumGU+T1-Myc was enriched for Hallmarks for E2F targets, G2M checkpoint, and interferon response pathways; in contrast, the group consisting of subtypes T1-TLum+T1-Early (and to some degree T1-Inflam) was characterized by activated SMAD3 and TP63 regulons, and enriched Hallmarks for TGF-Beta signaling, MYC targets, and oxidative phosphorylation (see FIG. 1). K) Differentially expressed genes were identified for T1-LumGU, with overexpressed genes shown by red dots and underexpressed by blue dots (see FIG. 11). This subtype had gene signatures suggestive of regulation by E2F and EZH2. Treatment of bladder cancer cell line HT-1376 with EPZ0011989 (Epizyme, Cambridge Mass.) resulted in increased expression of many of the repressed genes, depicted by orange dots, suggesting that subtype-specific genes may be regulated by EZH2. P values in A are from Fisher exact tests on contingency tables; those in E, F, and G are from Kruskal-Wallis tests on per-subtype FPKM distributions; all are uncorrected for multiple testing.

FIG. 2. Recurrence over time, with red highlighting patients that recurred within one year.

FIG. 3. Coarser- and finer-grained unsupervised consensus clustering solutions. A-C) Consensus membership heatmaps, and Kaplan-Meier plots for 24-mo recurrence, with log-rank p values, for A) 3-cluster, B) 4-cluster, and C) 5-cluster solutions.

FIG. 4. Per-subtype GSEA for CIS and inflammation gene sets. A) GSEA (CERNO) tests of CIS UP and DOWN gene sets (Supplemental Methods). B) Table of pathological CIS percentages and relative CIS level estimated from the AUCs, with a brief description of recurrences to 24 mo, by subtype. C) GSEA (CERNO) tests with a 170-gene inflammation gene set (Supplemental Methods).

FIG. 5. (A) Top GSEA (CERNO) tests on MSigDB 7.0 C3 motif gene sets for a signal-to-noise (StoN)-ordered set of 15853 coding genes. Above (‘DESC’): ‘enriched’ gene sets, i.e. sets that were enriched in genes that had a relatively high StoN value, i.e. were relatively highly expressed, in a subtype. ‘DESC’ refers to the left-to-right StoN sorting order of the 15853 genes. Below (‘ASC’): ‘repressed’ gene sets, i.e. sets that were enriched in genes that were relatively weakly expressed in a subtype. Tables give the MSigDB gene set ID and Title, then an AUC, and an adjusted p value from a CERNO test. B) Distributions of FPKM- and TPM-normalized expression for selected genes across T1 subtypes (Supplemental Methods). The two upper rows show the six genes in FIG. 1H.

FIG. 6. Top-ranked per-subtype GSEA results from CERNO tests on MSigDB Hallmark gene sets, using a signal-to-noise (StoN)-ordered set of 15853 coding genes. Above (‘DESC’): ‘enriched’ Hallmarks, i.e. gene sets that were enriched in genes that had a relatively high StoN value, i.e. were relatively highly expressed, in a subtype. Below (‘ASC’): ‘repressed’ Hallmarks, i.e. gene sets that were enriched in genes that were relatively weakly expressed in a subtype. Tables give the MSigDB gene set ID and Title, then AUCs that correspond to the tmod v0.40 evidencePlot graphic below, and an adjusted p value from a CERNO test. Below the tmod v0.40 evidencePlots, we show actual StoN profiles for each subtype in blue.

FIG. 7. Immune cell type deconvolution. A) MCPcounter, B) ESTIMATE, and C) CIBERSORT with the LM22 reference. P values are from Kruskal-Wallis tests, and are uncorrected for multiple testing.

FIG. 8. GSEA (CERNO) tests for Hallmark gene sets, comparing two sample groups in the discovery cohort: T1-Myc and T1-LumGU (S3+S1), and T1-TLum and T1-Early (S4+S5). A) Regulon status heatmap, as in FIG. 1A, with vertical grey boxes indicating the S3+S1 and S4+S5 sample groups. Horizontal boxes highlight the two dominant regulon activity status patterns (E2F1 activated/TP63 repressed, and the reverse of this). B) Selected MSigDB Hallmarks that were enriched in S3 and/or S1, or repressed in S4 and/or S5, were reported as enriched in S3+S1, relative to S4+S5 (FIG. 11). The blue signal-to-noise (StoN) profile below the AUC curves shows the profile that was used to sort 15853 coding genes for the GSEA calculations.

FIG. 9. Characterizing a 3-subtype solution for n=94 T1 non-cystectomy samples from the UROMOL cohort[7]. Distributions across subtypes of FPKM normalized gene expression for six coding genes. For the E2F1 graphic, brief text notes that subtype 3 had E2F1 and FOXM1 regulons activated, while subtype 1 had the ZNF385 regulon activated.

FIG. 10. Characterizing T1 subtypes in the early cystectomy validation cohort (n=26). A) Distributions of gene expression (FPKMs) for key genes. Subtype n=26-S1 has the E2F1 set of regulons activated and TP63 set repressed, while subtype n=26-S3 has the E2F1 set repressed and the TP63 set activated. P values are from Kruskal-Wallis tests, and are uncorrected for multiple hypothesis testing. For the E2F1 graphic, brief text notes that dES subtype 1 has the E2F1 regulon set activated, while subtype 3 has the TP63 regulon set activated. B) Summary of evidence supporting the predicted T1 subtypes in the validation cohort.

FIG. 11. Bladder cancer cell line experiments with an EZH2 inhibitor. A) Subtype-specific differentially expressed genes (DEGs) for the discovery cohort. B) For the HT-1376 bladder cancer cell line, DEGs for treatment with an EZH2 inhibitor vs. a DMSO control. Vertical lines indicate a threshold of |FC|>2.0, and the horizontal line a threshold of FDR=0.05.

FIG. 12. Quality control measures for RNA and RNA-seq data. A) Distributions of storage times in FFPE for the discovery and validation cohorts were not statistically different. B) Distributions of FFPE storage time across T1 subtypes. C) The core/noncore subtype membership metric was statistically uncorrelated to FFPE storage time. D) Read alignment rates were above 60% for all samples in the discovery cohort. E) The DV200 RNA quality metric was uncorrelated to the read alignment rate in the discovery cohort. F) Distribution of DV200 across the T1 subtypes.

FIG. 13. Characteristics of the five expression subtypes in the discovery cohort.

### DETAILED DESCRIPTION

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the claimed subject matter and does not pose a limitation on the scope of the claimed subject matter.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.

As used herein, a “subject in need thereof” may include a human and/or non-human animal. A “subject in need thereof” may include a subject having a disease or disorder associated with cell proliferative activity. A “subject in need thereof” may include a subject having a cell proliferative disease or disorder, which may include, but is not limited to cancer including bladder cancer.

As used herein, an “expression profile” means one or more values corresponding to a measurement of the relative abundance, level, presence, or absence of expression of a discriminative gene. An expression profile for a subject may be generated prior to or subsequent to a diagnosis of bladder cancer. Alternatively, an expression profile for a subject may be generated from a biological sample collected from the subject at one or more time points prior to or following treatment or therapy for bladder cancer. Alternatively, an expression profile for a subject may be generated from a biological sample collected from the subject at one or more time points during which there is no treatment or therapy (e.g., in order to monitor progression of disease or to assess development of disease in a subject having bladder cancer or at risk for developing bladder cancer). Alternatively, an expression profile for a subject may be generated from a biological sample collected from a healthy subject.

The methods and systems disclosed herein may be utilized in various aspects, for example, to classify bladder cancer from a subject (e.g., Stage T1 bladder cancer). The disclosed methods and systems may include one or more detecting steps such as steps for detecting expression of one or more genes in a biomarker panel. The methods may be performed in order to generate an expression profile for a sample of bladder cancer, which profile is utilized to classify the bladder cancer.

Biological samples may be utilized in the disclosed methods including samples of bladder tissue. The biological samples used in the disclosed methods may be isolated from a bladder biopsy. In some embodiments, the sample is a bladder tissue sample that is embedded in paraffin wax. Formalin fixation and tissue embedding in paraffin wax is a common approach for tissue sampling and storage. A major advantage of formalin-fixed paraffin-embedded (FFPE) specimens is the preservation of cellular and architectural morphologic detail. Methods are known in the art for the isolation of RNA from FFPE tissue.

In some embodiments of the disclosed methods, after mRNA is obtained from a sample, the mRNA converted to complementary DNA (cDNA). The cDNA then may be amplified, for example, by the polymerase chain reaction (PCR) or other amplification methods known to those of ordinary skill in the art. In some embodiments, cDNA is amplified with primers that introduce an additional DNA sequence (e.g., 3′ adenylation, adapter, reporter, capture sequence or moiety, barcode) onto the fragments (e.g., with the use of adapter-specific primers. The amplified DNA then may be sequenced in order to identify the corresponding mRNA and determine an expression level for the corresponding mRNA.

In some embodiments of the disclosed methods, an expression level of an RNA transcript or its expression product, is determined by normalization to the level of reference RNA transcripts or their expression products, which can be all measured transcripts (or their products) in the sample or a particular reference set of RNA transcripts (or their non-natural cDNA products). Normalization may be performed to correct for or normalize away both differences in the amount of RNA or cDNA assayed and variability in the quality of the RNA or cDNA used.

In some embodiments, microarrays are used to detect expression levels. An additional method of detecting expression levels is the use of a sequencing method, for example, RNAseq, next generation sequencing, and massively parallel signature sequencing (MPSS). These methods may be utilized to provide expression levels for thousands of genes in a cDNA library prepared from a sample.

The methods set forth herein provide a method for classifying a bladder cancer from a subject. After the expression levels are determined for one or more selected genes, for example by measuring non natural cDNA biomarker levels or non-natural mRNA-cDNA biomarker complexes, the biomarker levels are compared to reference values or a reference sample, for example with the use of statistical methods or direct comparison of detected levels, in order to classifying the bladder cancer.

Methods for comparing detected expression levels of biomarkers (e.g., genes) to reference values and/or reference samples also are provided herein. Based on the disclosed comparisons, a correlation between the biomarker expression levels obtained from the subject's bladder cancer sample and the reference values is obtained. A classification of the subject's bladder cancer sample then is made.

Results of the gene expression profile from a sample from a subject (test sample) may be compared to a biological sample(s) or data derived from a biological sample(s) that is known or suspected to be normal (“reference sample” or “normal sample”). In another embodiment, a reference sample or reference biomarker level data is obtained or derived from an individual known to have a bladder cancer subtype or classification as disclosed herein. The reference sample may be assayed at the same time, or at a different time from the test sample. Alternatively, the biomarker level information from a reference sample may be stored in a database or other means for access at a later date.

The biomarker level results of an assay on the test sample may be compared to the results of the same assay on a reference sample. In some cases, the results of the assay on the reference sample are from a database, or a reference value(s). In some cases, the results of the assay on the reference sample are a known or generally accepted value or range of values by those skilled in the art. In some cases the comparison is qualitative. In other cases the comparison is quantitative.

In one embodiment, a specified statistical confidence level may be determined in order to provide a confidence level regarding the bladder cancer classification. For example, it may be determined that a confidence level of greater than 90% may be a useful predictor of the bladder cancer classification. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen. The confidence level provided may in some cases be related to the quality of the sample, the quality of the data, the quality of the analysis, the specific methods used, and/or the number of gene expression values (i.e., the number of genes) analyzed.

Determining the bladder cancer classification in some cases be improved through the application of algorithms designed to normalize and or improve the reliability of the biomarker level data. In some embodiments of the present invention, the data analysis utilizes a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a biomarker level profile or profiles, e.g., to determine the bladder cancer classification. belongs.

In some cases the results of the biomarker level profiling assays, are entered into a database for access by representatives or agents of a molecular profiling business, the individual, a medical provider, or insurance provider. In some cases, assay results include sample classification, identification, or diagnosis by a representative, agent or consultant of the business, such as a medical professional. In other cases, a computer or algorithmic analysis of the data is provided automatically. In some cases the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the biomarker level profiling assays are presented as a report on a computer screen or as a paper record. In some embodiments, the report may include, but is not limited to, such information as one or more of the following: the levels of biomarkers as compared to the reference sample or reference value(s); the bladder cancer classification, and proposed therapies.

The disclosed biomarkers analyzed in the present methods and the expression levels thereof may be associated with various biological phenomena. In some embodiments, the expression levels assessed in the disclosed methods are associated with inflammation and/or the presence of carcinoma in situ (CIS)).

In some embodiments of the disclosed methods, the expression levels of a set of genes associates with inflammation may be analyzed. In some embodiments, the set of genes includes one or more of the following genes: MIR650, FCGR3A, IDO1, ALOX5AP, SLC15A3, C3AR1, ELK2AP, HAVCR2, PILRA, EVI2A, IGJ, C1R, IF130, CD3E, LTB, PTPRC, PLA2G7, RASSF4, IL4I1, CYTH4, IGLL5, HLA-DPA1, ITGB2, CPVL, HLA-DMA, ACP5, CD79A, NCKAP1L, GZMH, ADAP2, CXCL10, CD74, GZMB, GNLY, CD37, CD4, WIPF1, IL10RA, TRPV2, ABI3, CXCL9, HLA-DQB1, TYROBP, NKG7, CCL3, HCK, RNASE6, MSR1, CCR1, ITGAX, LYZ, SERPING1, IL2RG, CD2, TREM2, LCP2, CD48, F13A1, SAMSN1, RCSD1, UBD, HLA-DQA2, LAPTM5, GZMA, MS4A6A, CLEC7A, HCST, SASH3, IGSF6, TBXAS1, CIS, TMEM176A, C2, CD3D, CD7, EVI2B, CORO1A, SLAMF8, SLC7A7, LY86, C1QB, TMEM176B, CCL4, NCF2, FYB, SDS, SLAMF7, SLA, CD86, DOCK2, CXCL13, CCL5, SRGN, CYBB, SLCO2B1, HLA-DQB2, Clorfl62, LST1, WAS, MYO1F, C1QC, HLA-DMB, CD52, VSIG4, WARS, FGL2, PRF1, GZMK, STAB1, PIK3AP1, HLA-DRB1, RGS1, FPR3, GBP5, MS4A4A, SELPLG, MNDA, BCL2A1, CD300A, PTPN7, HLA-DRA, FCGR2A, RNASE1, SPIl, AIF1, C5AR1, CCL13, CYTIP, SPOCK2, ITGAM, HLA-DRB5, CD14, CD53, CCL19, CST7, FAM26F, LAIR1, LAG3, CD8A, ADAMDEC1, CCL21, HLA-DRB6, MZB1, FOLR2, FPR1, HLA-DOA, IL2RB, CIITA, S100B, OSCAR, ClQA, FCER1G, CD163, IL7R, LY96, PLEK, IRF8, FERMT3, TNFSF13B, HK3, HLA-DQA1, HLA-DPB1, CXCL11, CSF1R, MAFB, GPR183, MPEG1, DOK2, ARHGAP9, and LAT2.

In some embodiments of the disclosed methods, the expression levels of a set of genes associates with CIS may be analyzed (i.e., “CIS genes”). Genes whose expression should be higher in CIS may include but are not limited to: AKR1B10, CALD1, CDH11, CLIC4, COL15A1, COL3A1, CXCR4, DCN, DPYSL2, EFEMP1, FLNA, HLA-DQA1, HLA-DQB1, HOXA9, ITM2A, KPNA2, KYNU, LHFPL6, LUM, LYZ, MAN1C1, MSN, NR3C1, PDGFC, RARRES1, S100A8, SGCE, SPARC, TOP2A, TUBB, and UAP1. Genes whose expression should be lower in CIS may include but are not limited to: ACSBG1, ANXA10, BBC3, BCAM, BMP7, BST2, CA12, CLCA4, CRTAC1, CTSE, CYP2J2, EEF1A2, ENTPD3, FABP4, FGFR3, GRB7, HBG1, HOXA1, HOXB2, INA, ITGB4, IVL, KCNQ1, LAD1, LAMB3, LTBP3, MAPRE3, MST1R, PADI3, PLA2G2A, SOX15, TMPRSS4, TNNI2, TRIM29, UPK2, and UPK3B. The disclosed methods may include assessing the expression levels of one or more of the foregoing genes.

In some embodiments of the disclosed methods, the activation state of a regulon may be determined as a criteria for classifying a bladder cancer sample. In order to assess regulons, software (e.g., RTN v.2.11.6) may be utilized to generate a transcription network using coding expression data.

In some embodiments of the disclosed methods, the methods include detecting immune cells in a sample of bladder cancer. The detecting methods may include performing immune cell type deconvolution. The detecting methods may include determining expression values for MCPcounter markers. The detecting methods may include determining expression values for ESTIMATE markers. The detecting methods may include determining expression values for CIBERSORT markers.

In some embodiments of the disclosed methods, the methods include determining somatic copy number. Somatic copy number alterations may be estimated based on comparison to a reference genome.

### ILLUSTRATIVE EMBODIMENTS

The following Embodiments are illustrative and should not be interpreted to limit the scope of the claimed subject matter.

The disclosed subject matter relates to methods and systems for diagnosing, staging, stratifying, prognosing, and/or treating bladder cancer. In particular, the invention relates to methods and systems for classifying Stage T1 bladder cancer.

In some embodiments, the disclosed subject matter relates to methods for classifying a sample of Stage T1 bladder cancer from a subject. The disclosed methods may include performing one or more steps such as the following: the method comprising performing one or more of the following detecting steps: (a) detecting activation and/or repression in the sample of one or more regulons selected from E2F1, TP63, and ZNF385A; (b) detecting an increase in somatic copy number; (c) detecting expression in the sample of one or more genes selected from FOXM1, RXRA, MYC, E2F1, EZH2, FGFR3, STAT4, and NFATC2; (d) detecting tumor purity for the sample; (e) detecting an immune score and/or a stromal score for the sample; (f) detecting immune cells in the sample; (g) detecting inflammation in the sample; (h) detecting carcinoma in situ (CIS) in the sample, and/or detecting expression and/or repression of genes associated with CIS in the sample; (i) detecting expression of one or more genes selected from E2F target genes, G2M checkpoints genes, inflammatory response genes, IL2/STAT5 signaling genes, IFNG response genes, INFA response genes, and MYC target genes; and optionally performing the following classifying step: (j) classifying the sample by one or more subtype classifiers selected from Lund, TCGA mRNA subtype classifier, consensusMIBC, and UROMOL class; thereby classifying the Stage T1 bladder cancer.

In some embodiments of the disclosed methods, the methods comprise performing two, three, four, five, six, seven, eight, or nine or more of steps (a)-(j). In some embodiments, the methods comprise performing all ten steps (a)-(j).

The disclosed methods may be performed in order to classify a Stage T1 bladder cancer as belonging to a defined class. In some embodiments, the disclosed methods comprise classifying the Stage T1 bladder cancer into one of three, four, or five different classes. In some embodiments, the disclosed methods comprise classifying the Stage T1 bladder cancer into one of five (5) classes as follows: class 1 (which optionally may be referred to as “T1-LumGU”); class 2 (which optionally may be referred to as “T1-Inflam”), class 3 (which optionally may be referred to as “T1-Myc”), class 4 (which optionally may be referred to as “T1-TLum”), and class 5 (which optionally may be referred to as “T1-Early”).

In some embodiments, the disclosed methods may comprise classifying a Stage T1 bladder into a class 1, which optionally may be referred to as “T1-LumGU”) and may be defined by one or more of the following criteria: (a) activation of the E2F1 regulon relative to class 2, class 3, class 4, and/or class 5; (b) a high increase in somatic copy number relative to class 2, class 4, and/or class 5; (c) relatively high expression of E2F1 and EZH2 in comparison to class 2, class 3, class 4, and/or class 5; and relatively low expression of FGFR2 and MYC in comparison to class 2, class 3, class 4, and/or class 5; (d) high tumor purity; (e) a moderate immune score and/or a low stromal score; (h) presence of CIS, and/or increased or decreased expression of genes associated with CIS; (i) enriched expression of E2F target genes and/or G2M checkpoints genes relative to class 2, class 3, class 4, and/or class 5; and (j) a classification selected from Lund:GU, TCGA mRNA:Lum-papillary, consensus MIBC:LumU, and UROMOL class:2a. In some embodiments, class 1 may be defined by two, three, four, five, six, or seven criteria of (a), (b), (c), (d), (e), (h), (i), and (j). In some embodiments, class 1 may be defined by all eight of criteria (a), (b), (c), (d), (e), (h), (i), and 0).

In some embodiments, the disclosed methods may comprise classifying a Stage 1 bladder into a class 2, which optionally may be referred to as “T1-Inflam”) and may be defined by one or more of the following criteria: (a) activation of the TP63/ZNF385A regulon relative to class 1, class 3, and/or class 5; (b) a low increase in somatic copy number relative to class 1; (c) relatively high expression of STAT4 and NFATC2 in comparison to class 1, class 3, class 4, and/or class 5; (d) low tumor purity relative to class 1, class 3, class 4, and/or class 5; (e) a high immune score and/or a high stromal score relative to class 1, class 3, class 4, and/or class 5; (f) detected immune cells; (g) detected inflammation; (h) presence of CIS, and/or increased or decreased expression of genes associated with CIS; (i) enrichment in expression of inflammatory response genes, IL2/STAT5 signaling genes, and IFNG response genes, and MYC target genes; and repression of expression of E2F target genes, G2M checkpoint genes, and MYC target genes relative to class 1, class 3, and/or class 5; and (j) a classification selected from Lund:(URO, some Basal/SCC-like, Mes-like), TCGA mRNA:Lum-papillary, consensusMIBC:LumP/Stroma-rich, and UROMOL class:2a/2b. In some embodiments, class 2 may be defined by two, three, four, five, six, seven, eight, or nine of criteria of (a), (b), (c), (d), (e), (f), (g), (h), (i), and 0). In some embodiments, class 2 may be defined by all ten of criteria (a), (b), (c), (d), (e), (f), (g), (h), (i), and 0).

In some embodiments, the disclosed methods may comprise classifying a Stage 1 bladder into a class 3, which optionally may be referred to as “T1-Myc”) and may be defined by one or more of the following criteria: (a) moderate activation of the E2F1 regulon relative to class 1, class 2, class 4, and/or class 5; (c) relatively high expression of FOXM1 and RXRA in comparison to class 1, class 2, class 4, or class 5, and a wide range of expression for MYC in comparison to class 1, class 2, and/or class 4; (d) high tumor purity; (e) a low immune score and/or a low stromal score; (h) presence of CIS, and/or increased or decreased expression of genes associated with CIS; and 0) a classification selected from Lund:URO, TCGA mRNA:Lum-papillary, consensusMIBC:LumP, and UROMOL class:2a. In some embodiments, class 3 may be defined by two, three, four, or five of criteria of (a), (c), (d), (e), (h), and 0). In some embodiments, class 3 may be defined by all six of criteria (a), (c), (d), (e), (h), and ( ).

In some embodiments, the disclosed methods may comprise classifying a Stage 1 bladder into a class 4, which optionally may be referred to as “T1-TLum” and may be defined by one or more of the following criteria: (a) activation of the ZNF385A (TP63) regulon relative to class 1, class 2, and/or class 5; (b) a low increase in somatic copy number relative to class 1, class 2, and/or class 5; (c) relatively low expression of E2F1, FOXM1, STAT4, and NFATC2 in comparison to class 1, class 2, class 3, and/or class 5; (d) high tumor purity; (e) a low immune score and/or a low stromal score; (h) presence of CIS, and/or increased or decreased expression of genes associated with CIS; (i) repression of expression E2F target genes and G2M checkpoint genes relative to class 1, class 3, and/or class 5; and (j) a classification selected from Lund:URO, TCGA mRNA:Lum-papillary, consensusMIBC:LumP, and UROMOL class:1/2a. In some embodiments, class 4 may be defined by two, three, four, five, six, or seven of criteria of (a), (b), (c), (d), (e), (h), (i), and (j). In some embodiments, class 4 may be defined by all eight of criteria (a), (b), (c), (d), (e), (h), (i), and (j).

In some embodiments, the disclosed methods may comprise classifying a Stage 1 bladder into a class 5, which optionally may be referred to as “T1-Early” and may be defined by one or more of the following criteria: (a) moderate activation of the ZNF385A regulon relative to class 1, class 2, class 3, and/or class 4; (b) a low increase in somatic copy number relative to class 1; (c) relatively high expression of MYC in comparison to class 1, class 2, or class 4; (d) high tumor purity; (e) a low immune score and/or a low stromal score; (h) absence of CIS, and/or increased or decreased expression of genes associated with CIS in the sample; (i) enrichment in expression of MYC target genes; and repression of expression of IFNG genes and IFNA genes relative to class 1, class 2, and class 4; and (j) a classification selected from Lund:URO, TCGA mRNA:Lum-papillary, consensusMIBC:LumP, and UROMOL class:2a/3. In some embodiments, class 5 may be defined by two, three, four, five, six, or seven of criteria of (a), (b), (c), (d), (e), (h), (i), and (j). In some embodiments, class 5 may be defined by all eight of criteria (a), (b), (c), (d), (e), (h), (i), and (j).

The disclosed methods may include steps that include detecting expression of one or more genes in a sample. In some embodiments of the disclosed methods, detecting expression comprises detecting mRNA in a sample. In some embodiments of the disclosed methods, mRNA may be detecting in a sample by converting mRNA in a sample to cDNA (e.g., preparing a cDNA library from a sample) and detecting the cDNA. A cDNA library may be prepared by a method that includes one or more of the following steps: cDNA synthesis of an mRNA sample, 3′end adenylation, adapter ligation, and library PCR amplification. cDNA may be detecting (e.g., in a cDNA library) by methods that include but are not limited to sequencing.

The disclosed methods may include administering therapy for bladder cancer to the subject after classifying the sample of Stage 1 bladder cancer. Therapies that may be administered in the disclosed methods may include, but are not limited to: intravesical immunotherapy (e.g., Bacillus Calmette-Guerin (BCG) therapy), intravesical chemotherapy (e.g., mitomycin, or electromotive mitomycin therapy), gemcitabine, or valrubicin), systemic chemotherapy (e.g., cisplatin, fluorouracil (5-FU), mitomycin, gemcitabine, methotrexate, vinblastine, doxorubicin (e.g., Adriamycin), paclitaxel, docetaxel, ifosfamide, peptrexed), targeted RNA interference therapy (RNAi) (e.g., RNAi therapy against EZH2 (EZH2-i) or MYC (MYC-i)), bladder cancer surgery (e.g., cystectomy (e.g., partial or radical) or transurethral resection of a bladder tumor (TURBT)), nadofaragene firadenovec therapy (e.g., Adstiladrin® brand or Instiladrin® brand), radiation therapy, and systemic immunotherapy (e.g., PD-1 and PD-L1 inhibitors (e.g., atezolizumab, avelumab, nivolumab, or pembrolizumab).

In some embodiments of the disclosed methods, the subject sample is classified as class 1 (which optionally may be referred to as “T1-LumGU”), and the method includes administering therapy to the subject comprises administering BCG therapy and/or EZH2-i therapy to the subject.

In some embodiments of the disclosed methods, the subject sample is classified as class 2 (which optionally may be referred to as “T1-Inflam”), and the method includes administering therapy to the subject comprises administering BCG therapy to the subject.

In some embodiments of the disclosed methods, the subject sample is classified as class 3 (which optionally may be referred to as “T1-Myc”), and the method includes administering therapy to the subject comprises performing a cystectomy on the subject and/or administering Myc-I therapy to the subject.

In some embodiments of the disclosed methods, the subject sample is classified as class 4 (which optionally may be referred to as “T1-TLum”), and the method includes administering therapy to the subject comprises administering TURBT or BCG to the subject.

In some embodiments of the disclosed methods, the subject sample is classified as class 5, (which optionally may be referred to as “T1-Early”), and the method comprises administering therapy to the subject comprises administering nadofaragene firadenovec and/or MYC-i therapy to the subject.

Also disclosed herein are systems that are configured for performing one or more steps of the disclosed methods. In some embodiments, the disclosed system is utilized for classifying a sample of Stage 1 bladder cancer from a subject, and the system comprises a computer processor configured for receiving input obtained by performing one or more of the following detecting steps: (a) detecting activation and/or repression in the sample of one or more regulons selected from E2F1, TP63, and ZNF385A; (b) detecting an increase in somatic copy number; (c) detecting expression in the sample of one or more genes selected from FOXM1, RXRA, MYC, E2F1, EZH2, FGFR3, STAT4, and NFATC2; (d) detecting tumor purity for the sample; (e) detecting an immune score and/or a stromal score for the sample; (f) detecting immune cells in the sample; (g) detecting inflammation in the sample; (h) detecting carcinoma in situ (CIS) in the sample, and/or detecting expression and/or repression of genes associated with CIS in the sample; (i) detecting expression of one or more genes selected from E2F target genes, G2M checkpoints genes, inflammatory response genes, IL2/STAT5 signaling genes, IFNG response genes, INFA response genes, and MYC target genes; and optionally performing the following classifying step: j) classifying the sample by one or more subtype classifiers selected from Lund, TCGA mRNA subtype classifier, consensusMIBC, and UROMOL class; wherein the computer processor provides an output comprising a classification for the sample of Stage 1 bladder cancer based on the input.

### EXAMPLES

The following Examples are illustrative and should not be interpreted to limit the scope of the claimed subject matter.

### Example 1

Abstract

Stage T1 bladder cancers have the highest progression and recurrence rates of all non-muscle invasive bladder cancers (NMIBC). Most T1 cancers are treated with BCG, but many will progress or recur, and some T1 patients will die from bladder cancer. Particularly aggressive tumors could be treated by early cystectomy. To better understand the molecular heterogeneity of T1 cancers, we performed transcriptome profiling and unsupervised clustering, identifying five consensus subtypes of T1 tumors treated with reTUR, induction and maintenance BCG. The T1-LumGU subtype was associated with CIS (6/13, 46% of all CIS), had high E2F1 and EZH2 expression, and enriched E2F target and G2M checkpoint Hallmarks. T1-Inflam was inflamed and infiltrated with immune cells. While most T1 tumors were classified as luminal papillary, the T1-TLum subtype had the highest median Luminal Papillary score and FGFR3 expression, no recurrence events, and the fewest copy number gains. T1-Myc and T1-Early subtypes had the most recurrences (14/30 within 24 months), highest median MYC expression, and, when combined, had significantly worse recurrence-free survival than the other three subtypes. T1-Early had 5 (38%) recurrences within the first 6 months of BCG, and repressed gene sets for inflammation, and IFN-alpha and IFN-gamma Hallmarks. We developed a single-patient T1 classifier and validated our subtype biology in a second cohort of T1 tumors. Future research will be necessary to validate the proposed T1 subtypes and to determine if therapies can be individualized for each subtype.

Applications

Applications of the disclosed technology may include but are not limited to: (i) risk-stratification of patients with T1 bladder cancer; (ii) identification of the tumor biology of T1 bladder cancer; and (iii) identification of potential therapies for T1 bladder cancer, which include, but are not limited to, immunotherapy, EZH2 targeted therapy, anti-Myc therapy, IFNalpha therapy, BCG, or radical cystectomy.

Advantages

Applications of the disclosed technology may include but are not limited to: (i) by identifying the subtype of a patient's tumor, the disclosed classifier may help identify whether cystectomy or BCG is a more appropriate treatment for a patient; and (ii) each subtype of bladder cancer has a uniquely described biology, and may have a specific therapy that works better for that subtype

Brief Summary

Please see the enclosed manuscript entitled “Identification of Differential Tumor Subtypes of T1 Bladder Cancer,” which is provided as an Appendix that accompanies this application and forms part of this application. The subject matter disclosed in the Appendix is incorporated herein by reference in its entirety.

The manuscript summarizes the work done to identify different subtypes of T1 bladder cancer, and describes the characteristics of the subtypes in order to provide a classifier. The disclosed classifier will allow others to be able to identify these subtypes of T1 bladder cancer. The classifier is available online: https://github.com/csgroen/classifyT1BC.

### Example 2—Identification of Differential Tumor Subtypes of T1 Bladder Cancer

Reference is made to Robertson et al., “Identification of Differential Tumor Subtypes of T1 Bladder Cancer,” Eur. Urol., Volume 78, Issue 4, October 2020, Pages 533-537, the content of which is incorporated herein by reference in its entirety.

Patient Summary

We identify and characterize expression subtypes of T1HG bladder cancer that are biologically heterogeneous and have variable responses to BCG treatment. We validate subtypes, and describe a single-patient classifier.

Brief Correspondence

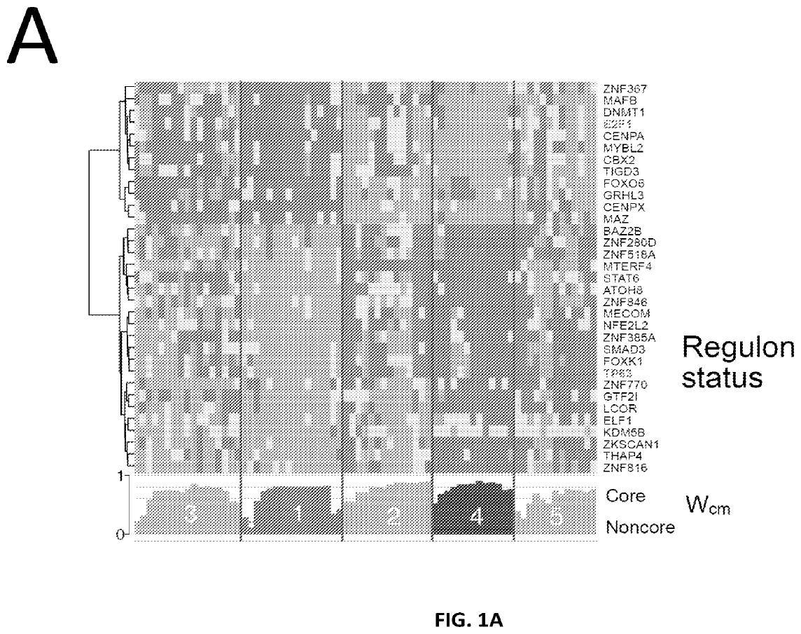

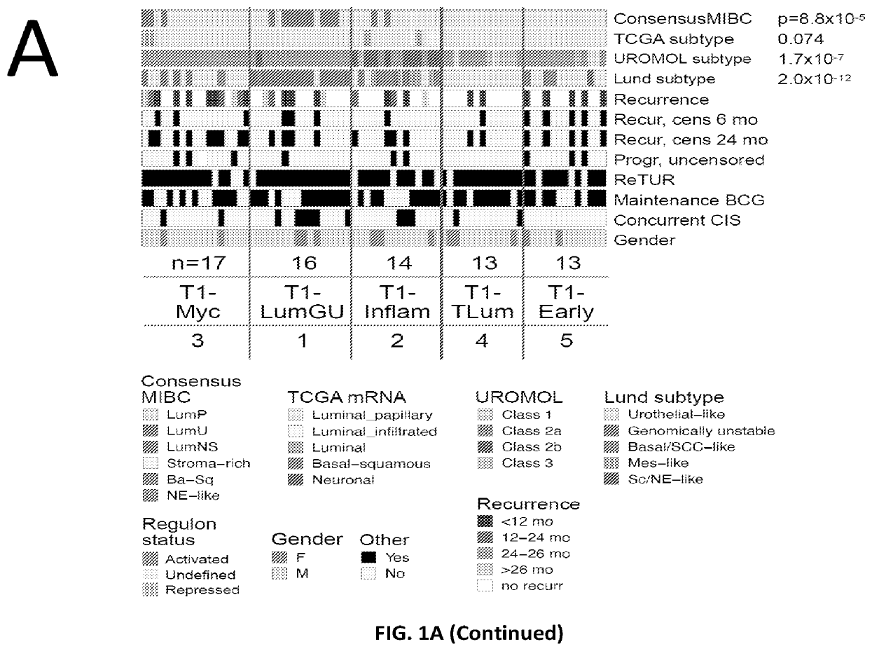

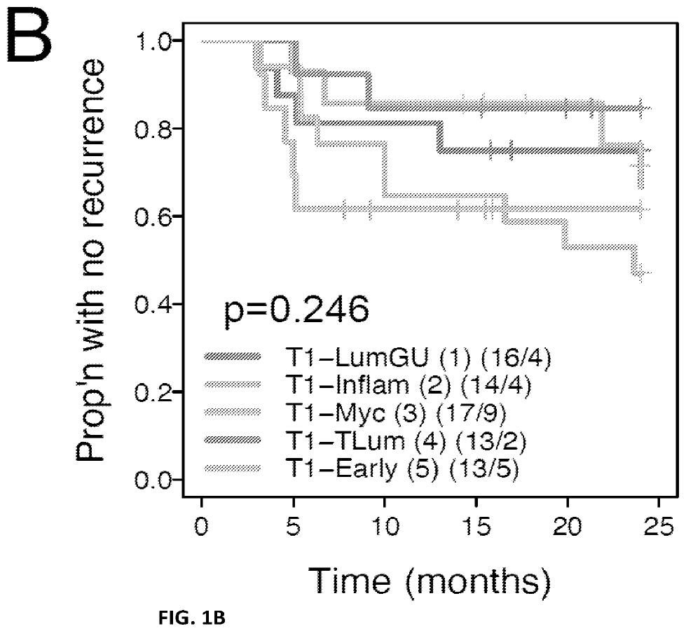

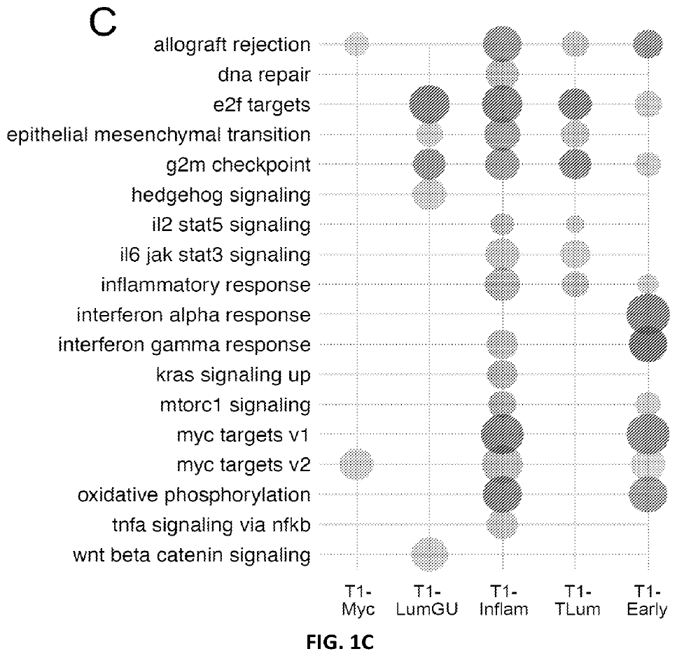

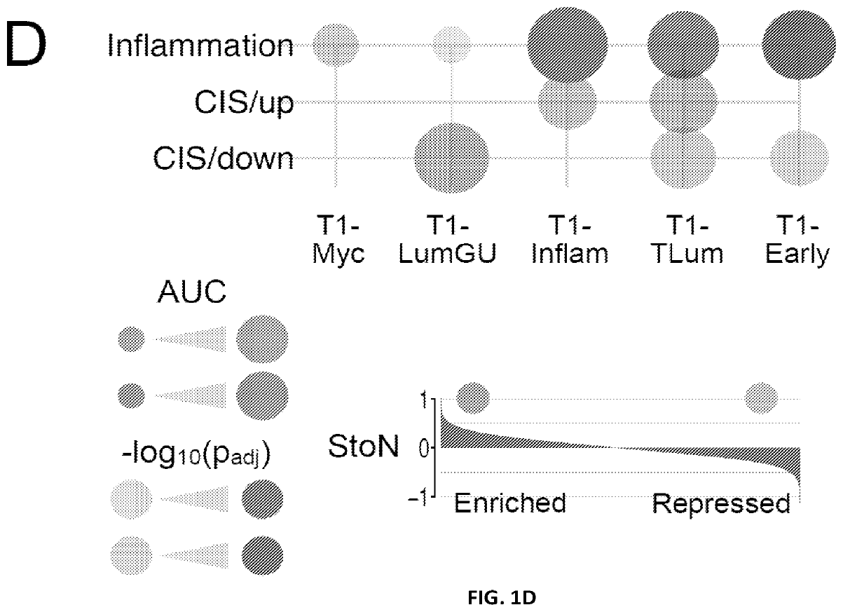

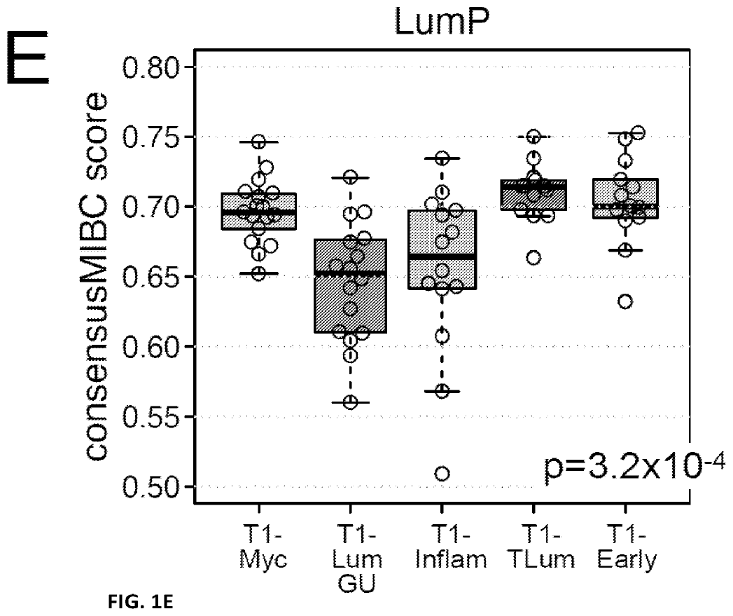

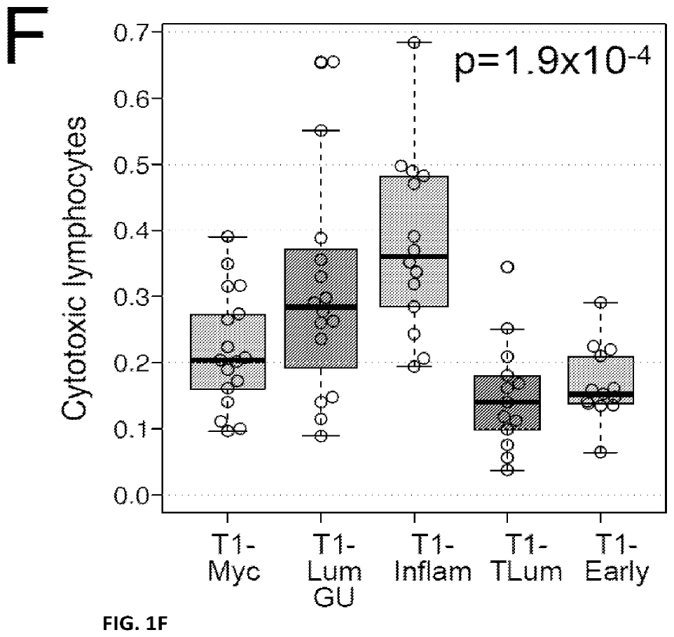

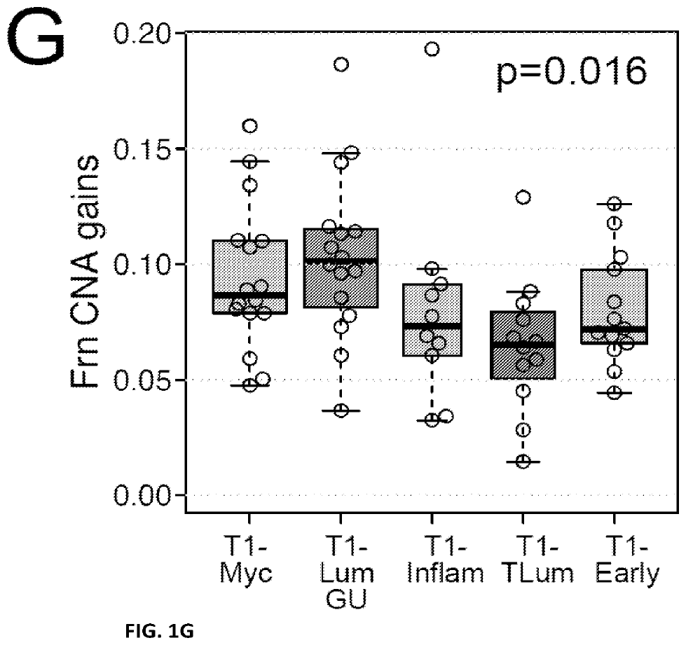

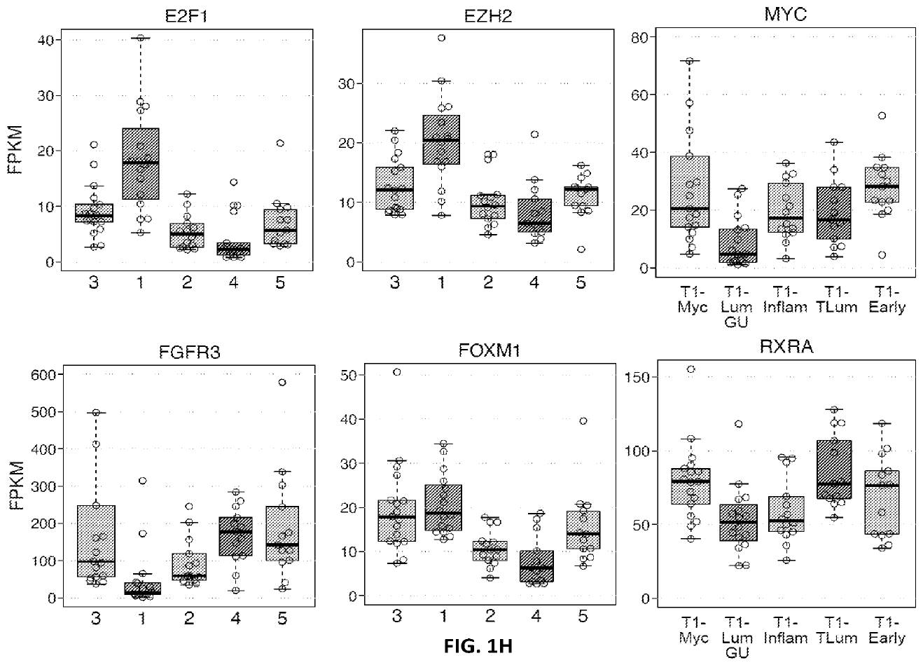

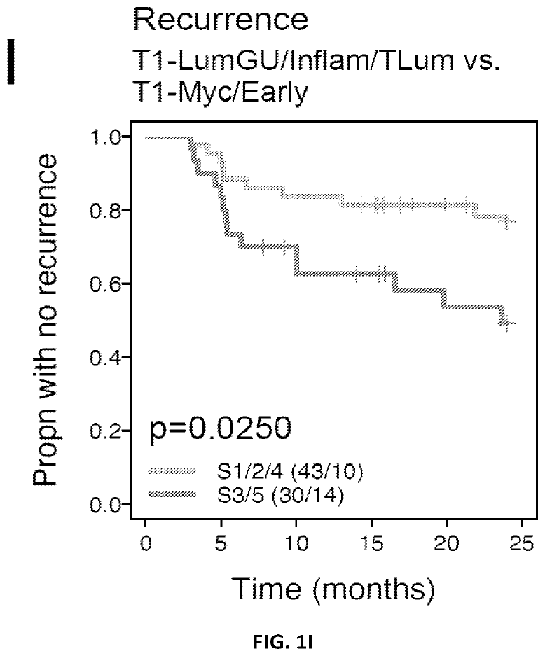

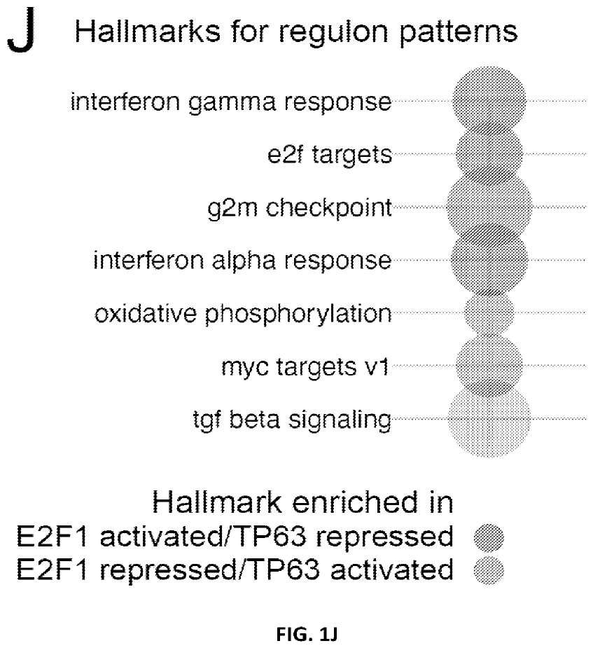

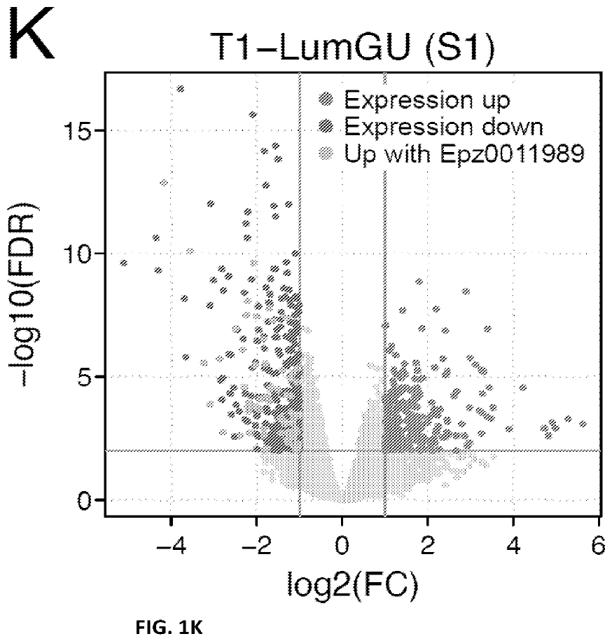

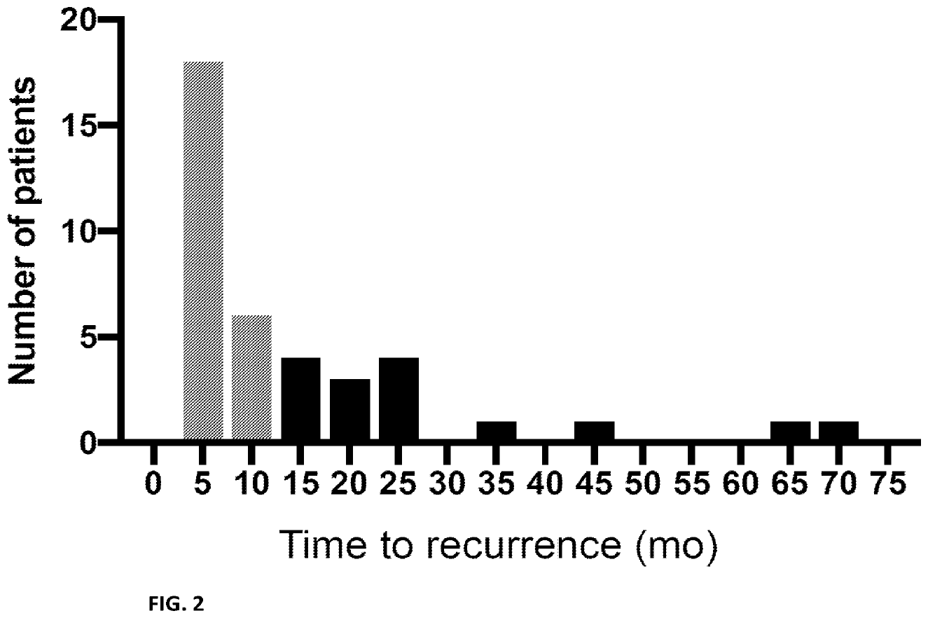

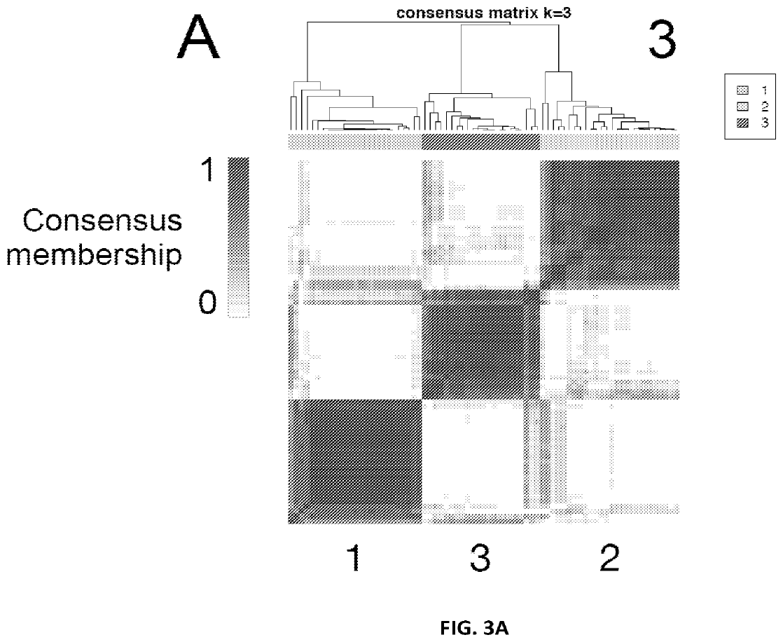

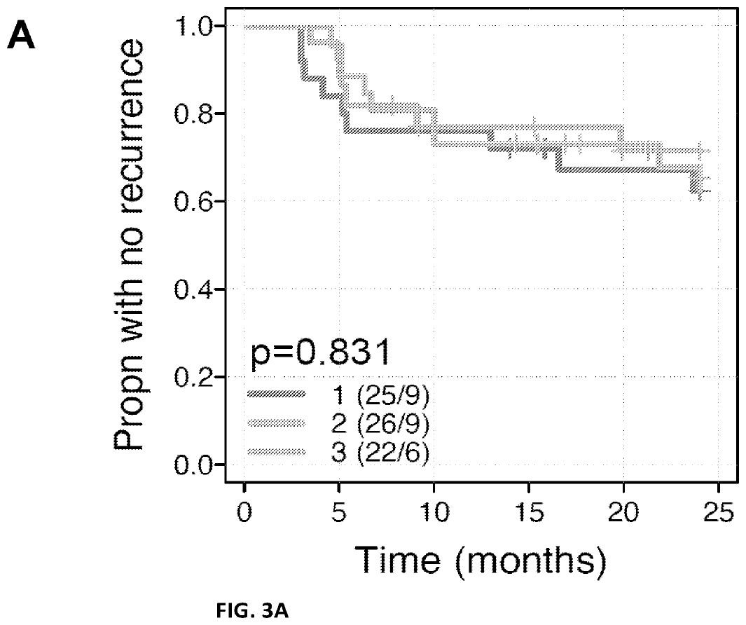

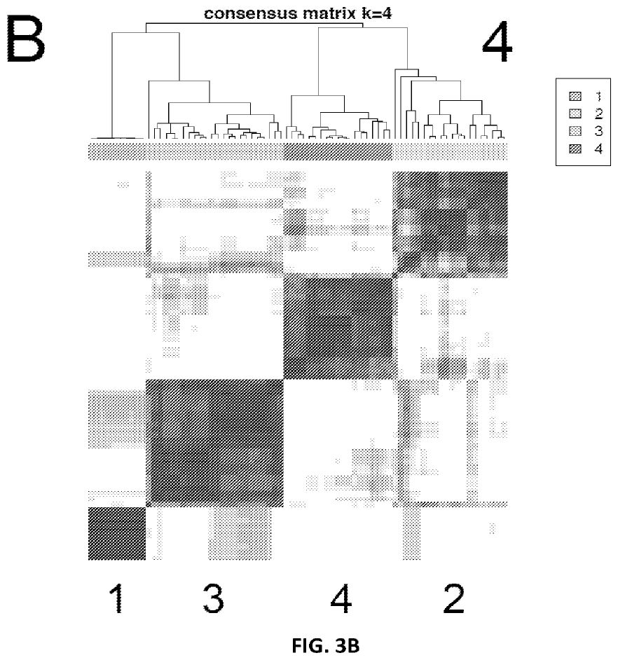

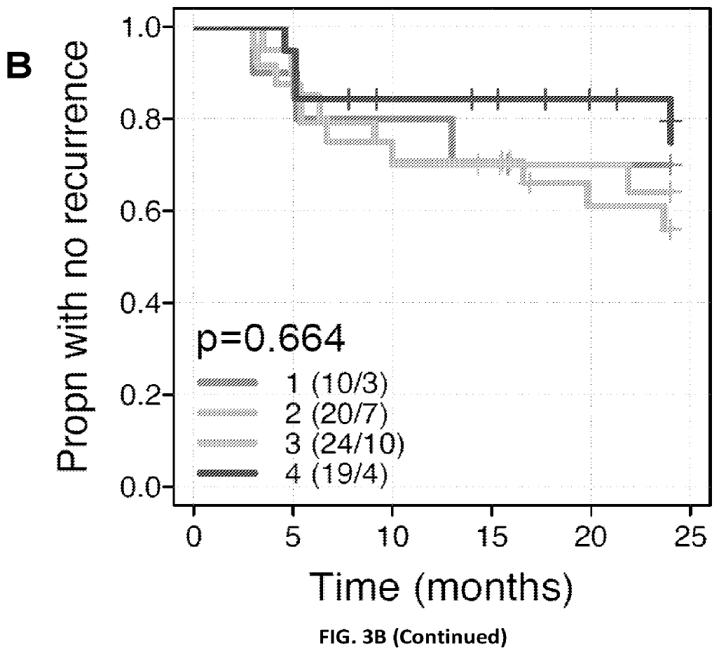

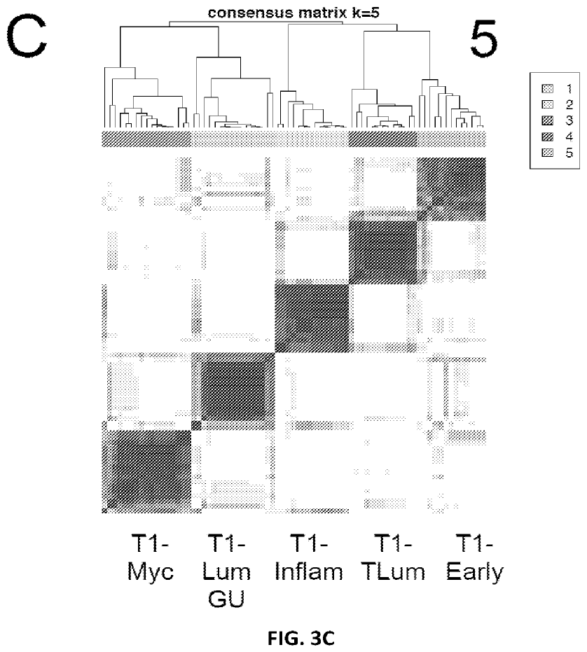

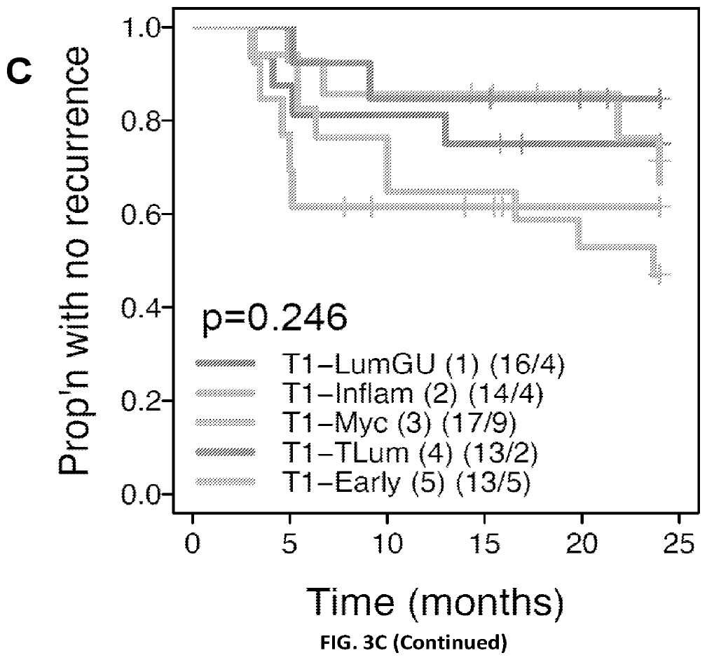

T1 tumors are potentially the most aggressive subtype of NMIBC, with 40% recurrence and 15% progression at 5 years [1]. While most T1 cancers are treated with BCG (Bacillus Calmette-Guerin), recurrence or progression is associated with radical cystectomy, with decreased survival secondary to delayed intervention [1]. Biomarkers that could predict response to BCG in T1 cancers could help both patients and clinicians make treatment-related decisions; however, few to no tumor-specific prognostic features have been identified. We previously reported that T1s and MIBCs had similar mutations, but that mutations were unable to predict response to BCG [2]. The primary objective of our study was to investigate the molecular heterogeneity of T1 cancers by RNA-Seq of 73 primary T1 tumors (Table 1A), with the primary endpoint of recurrence after BCG. To minimize sources of bias, all tumors were treated at the same institution, 84% had reTUR, and all received induction and maintenance BCG (64%) if they did not recur. Twenty-four-month recurrence was 32% (23/73), with 25% recurrence at one year (18), and 8% 24-month progression (6/73) (Table 1A, FIG. 2). We focused our analysis on tumor expression subtypes that we identified by unsupervised consensus clustering. After assessing 3, 4 and 5-cluster solutions (FIG. 3), we characterized a 5-cluster solution whose subtypes had distinct clinical outcomes and biological characteristics (FIG. 1), and we combined gene expression, gene-set enrichment, and regulon analyses to describe the distinct biology in each subtype (Supplemental Methods)[3].

TABLE 1
A

| Discovery cohort | | | Percent |
|---|---|---|---|
| Number of patients | | 73 | |
| Age (median) | | 73 yr | |
| Gender | Female | 13 | 18% |
| CIS | | 13 | 18% |
| Race | White | 57 | 78% |
| | Black | 5 | 7% |
| | Asian | 2 | 3% |
| | Other or unknown | 9 | 12% |
| reTUR | | 61 | 84% |
| Received maintenance BCG | | 47 | 64% |
| Recurrence | Within 6 mo | 13 | 18% |
| | Within 12 mo | 18 | 25% |
| | Within 24 mo | 23 | 32% |
| Progression | Within 24 mo | 6 | 8% |

B

| Validation cohort | | | Percent |
|---|---|---|---|
| Number of patients | | 26 | |
| Age (median) | | 71 yr | |
| Gender | Female | 8 | 31% |
| CIS | | 11 | 42% |
| Pathology | T1 or less | 18 | 69% |
| | T2 or greater | 8 | 31% |
| | N+ | 4 | 15% |
| | Progression or BCSD | 5 | 5% |

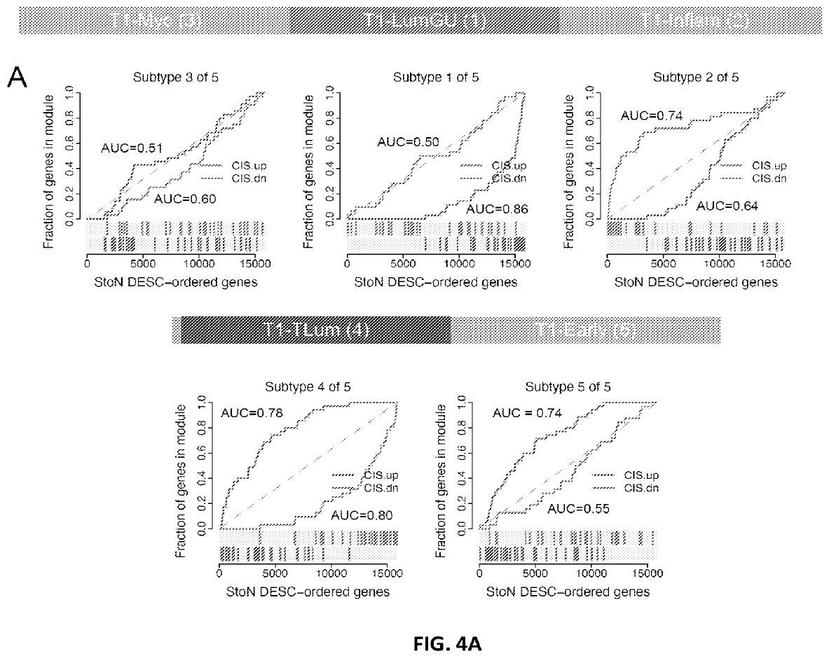

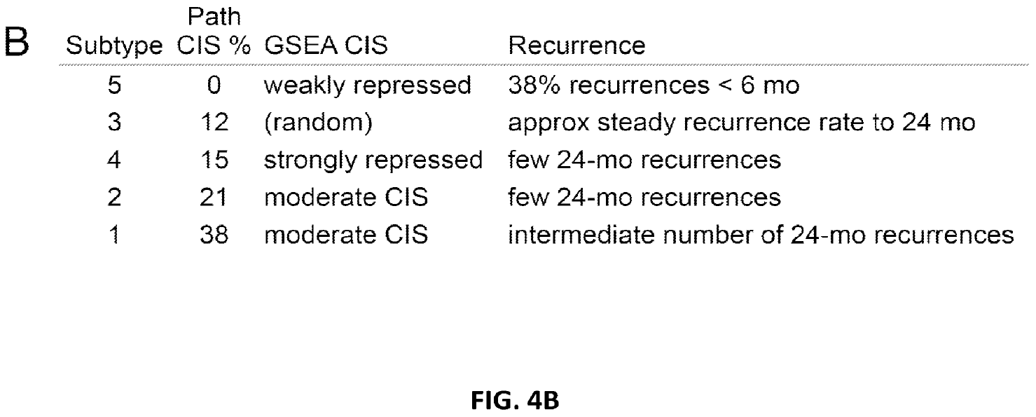

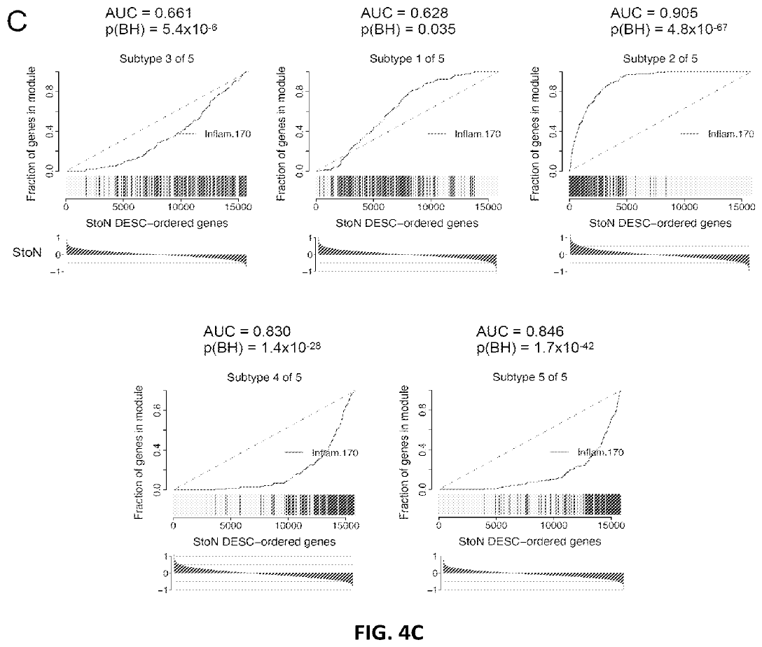

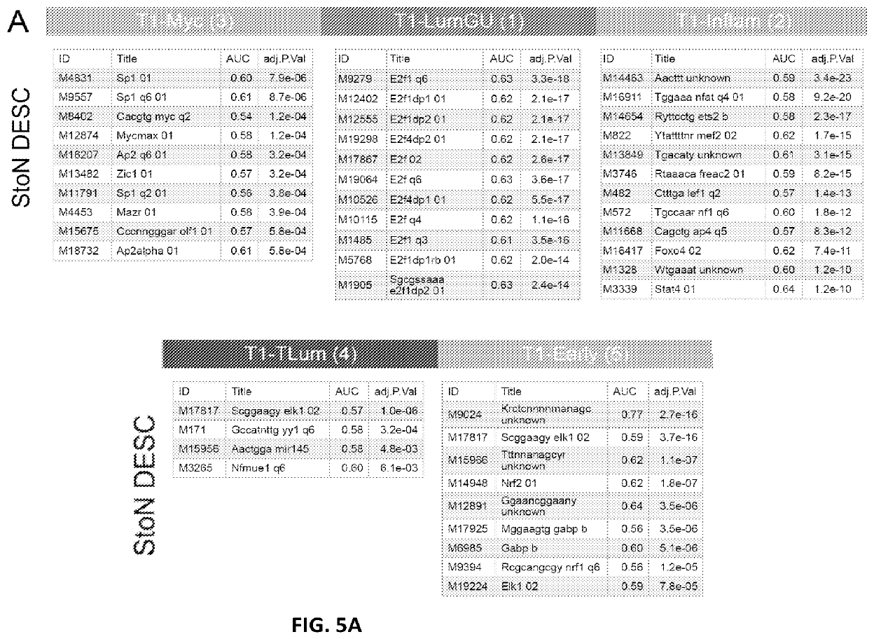

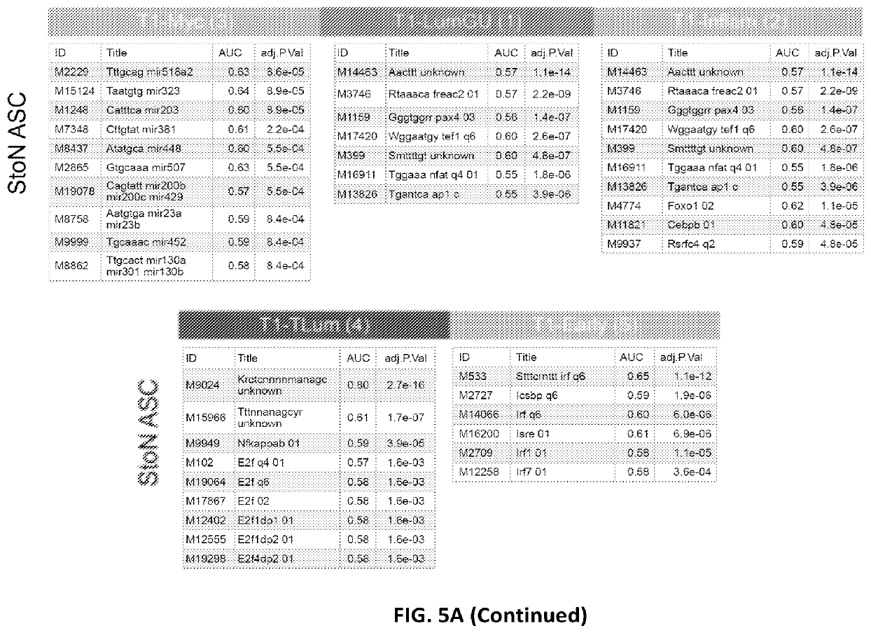

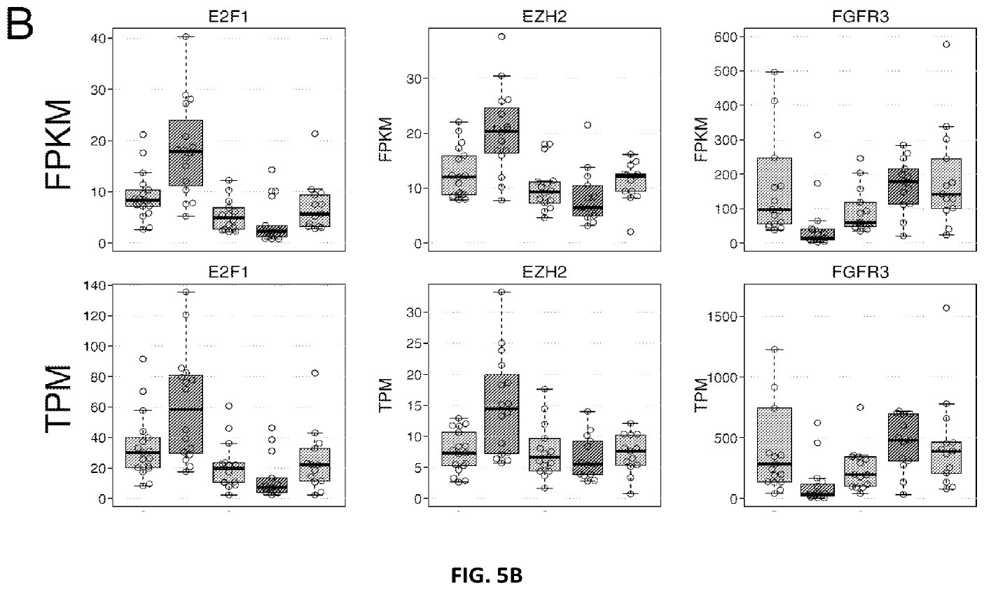

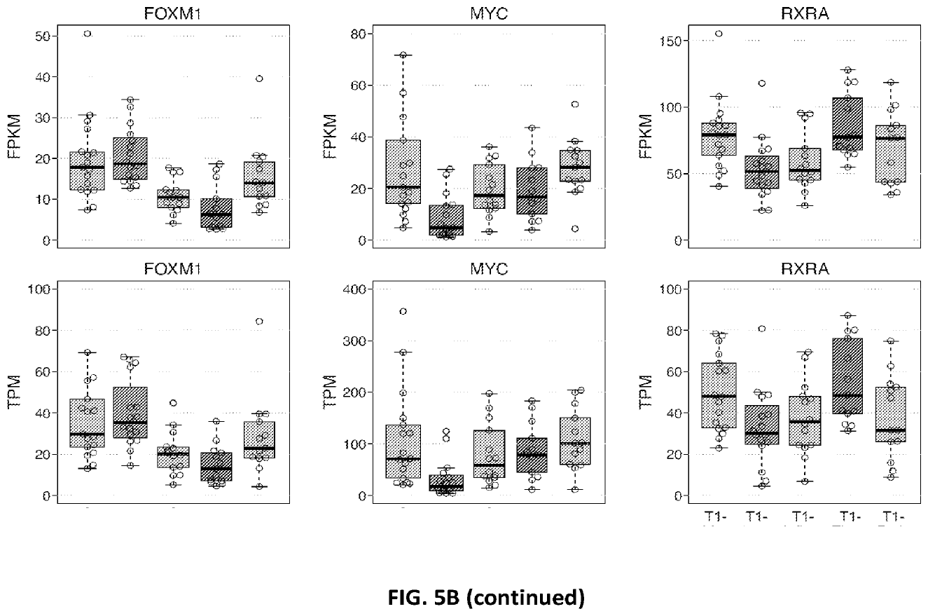

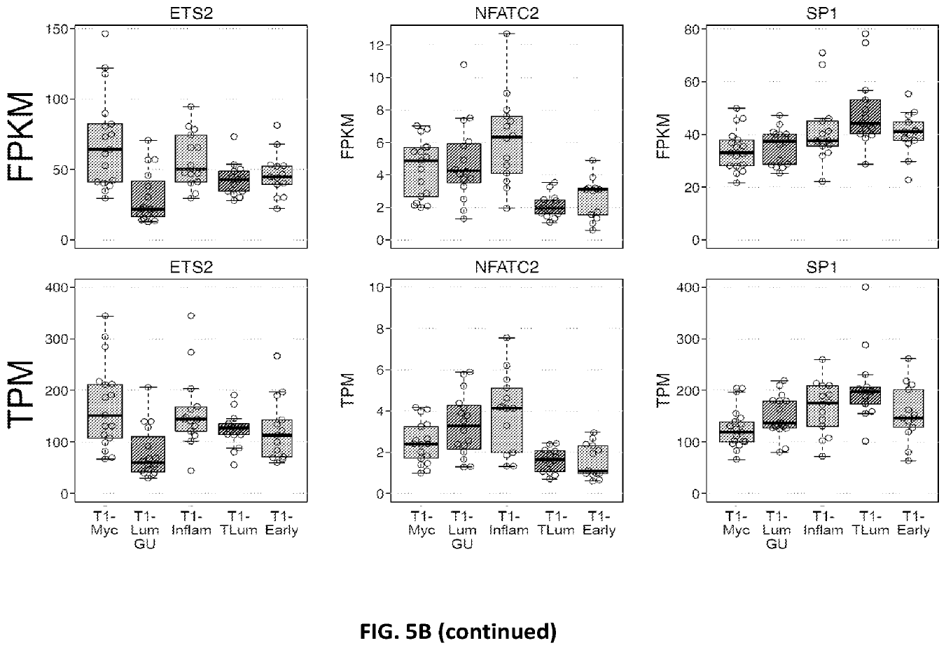

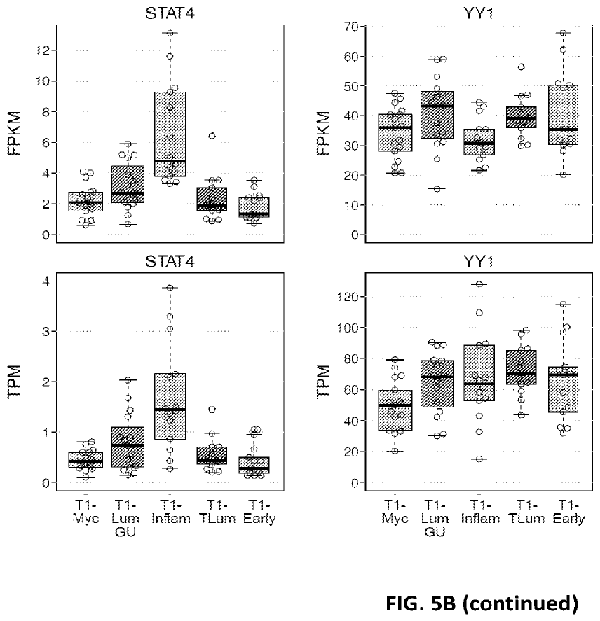

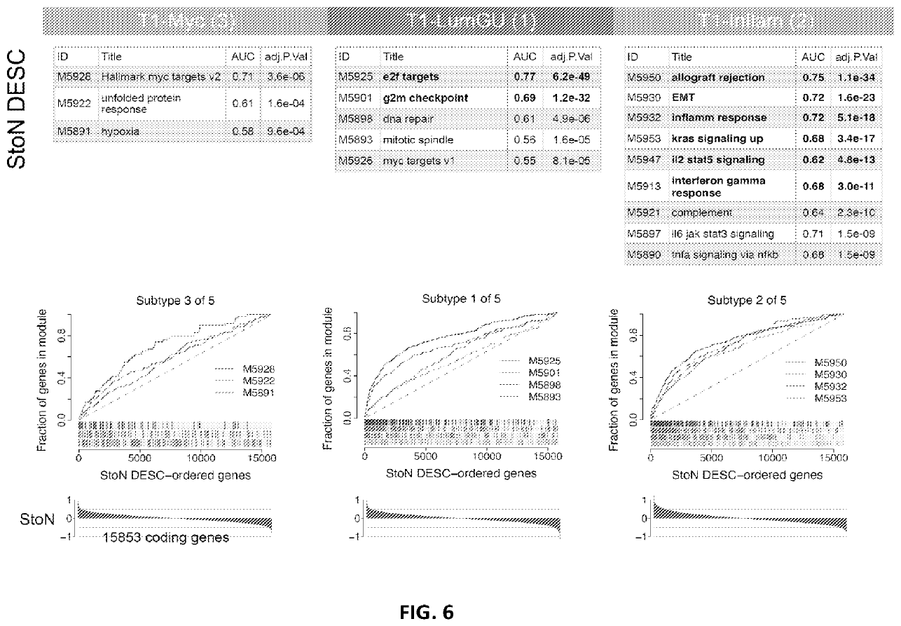

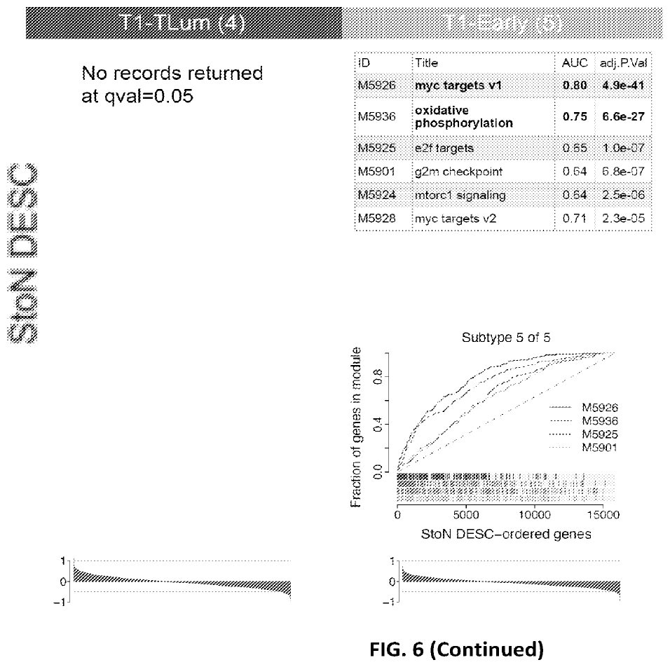

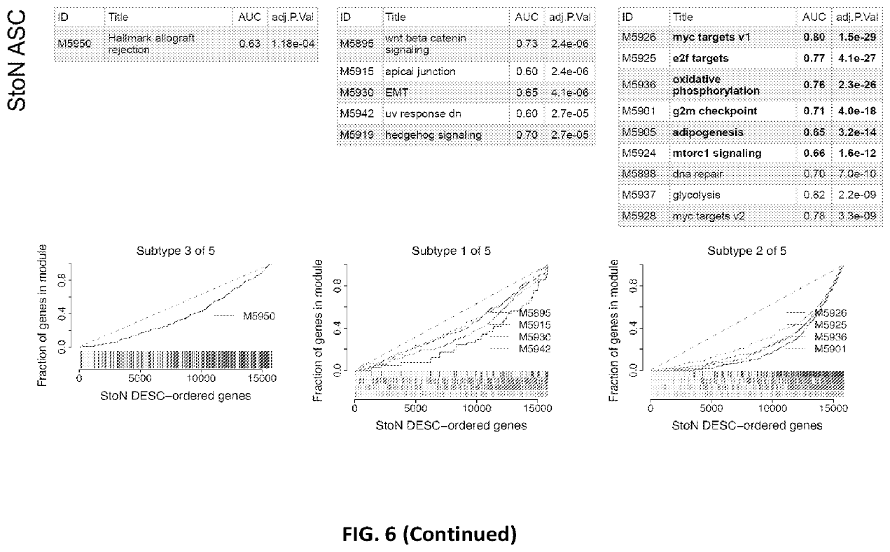

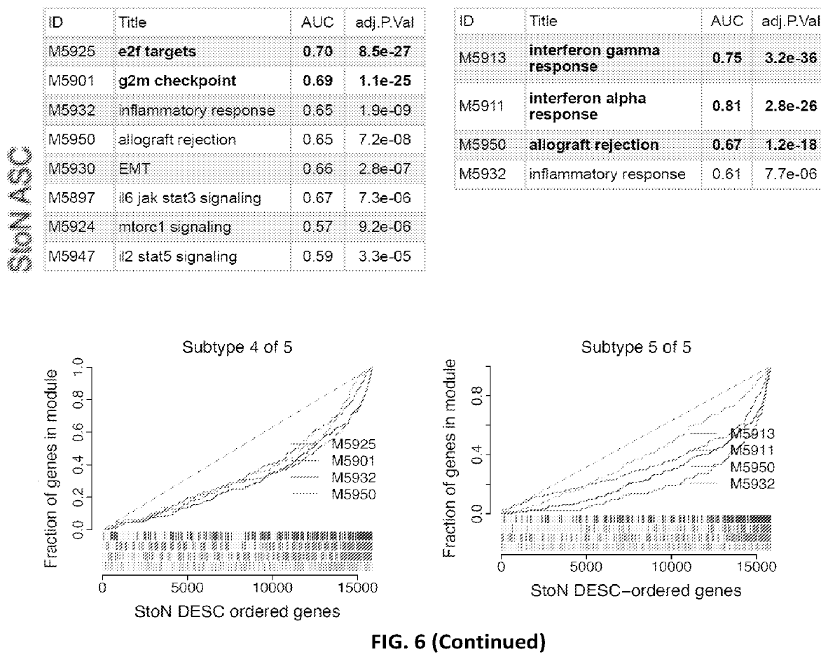

T1-LumGU (S1). The T1-Luminal Genomically Unstable (T1-LumGU) subtype had the highest frequency of pathologic CIS (6/16, 38% of T1-LumGU vs. 13/73, 18% of all samples) (Table 2) with moderate enrichment for CIS UP and DOWN gene sets, and a recurrence rate of 4/16 (25%) at 24 months (FIGS. 1D, 4A,B; Example 3—Supplementary Methods). Analysis of T1-LumGU by other classifiers found 14 (88%) of 16 samples to be Lund GU and 9 (56%) consensus MIBC LumU [4]. T1-LumGUs had the largest median number of somatic copy number (CN) gains (FIG. 1G, p=0.016, Kruskal test). While inflammation genes were weakly repressed in T1-LumGU tumors, MSigDB C3 E2F1 motifs were enriched (FIG. 5), as were Hallmark gene sets for E2F targets and G2M checkpoint (AUC=0.77 and 0.69, FIG. 6).

TABLE 2

| | | T1 subtype | | | | | |
|---|---|---|---|---|---|---|---|
| | | T1-Myc | T1-LumGU | T1-Inflam | T1-TLum | T1-Early | Sum |
| Pathological CIS | No CIS | 15 | 10 | 11 | 11 | 13 | 60 |
| vs T1 subtype | CIS | 2 | 6 | 3 | 2 | 0 | 13 |
| | Sum | 17 | 16 | 14 | 13 | 13 | 73 |
| | | T1 subtype | | | | | |
| | | T1-Myc | T1-LumGU | T1-Inflam | T1-TLum | T1-Early | Sum |
| TCGA MIBC | Luminal | 2 | 0 | 2 | 0 | 0 | 4 |
| mRNA subtype | Luminal_ | 0 | 0 | 3 | 0 | 0 | 3 |
| | infiltrated | | | | | | |
| | Luminal_ | 15 | 16 | 9 | 13 | 13 | 66 |
| | papillary | | | | | | |
| | Sum | 17 | 16 | 14 | 13 | 13 | 73 |
| | | T1 subtype | | | | | |
| | | T1-Myc | T1-LumGU | T1-Inflam | T1-TLum | T1-Early | Sum |
| consensusMIBC | Ba/Sq | 0 | 0 | 1 | 0 | 0 | 1 |
| subtype | LumNS | 2 | 0 | 1 | 0 | 0 | 3 |
| | LumP | 14 | 7 | 11 | 13 | 13 | 58 |
| | LumU | 1 | 9 | 0 | 0 | 0 | 10 |
| | Stroma- | 0 | 0 | 1 | 0 | 0 | 1 |
| | rich | | | | | | |
| | NE-like | 0 | 0 | 0 | 0 | 0 | 0 |
| | Sum | 17 | 16 | 14 | 13 | 13 | 73 |
| | | T1 subtype | | | | | |
| | | T1-Myc | T1-LumGU | T1-Inflam | T1-TLum | T1-Early | Sum |
| UROMOL 2 | Class_1 | 0 | 0 | 1 | 4 | 1 | 6 |
| class | Class_2a | 17 | 15 | 4 | 7 | 10 | 53 |
| | Class_2b | 0 | 1 | 9 | 0 | 0 | 10 |
| | Class_3 | 0 | 0 | 0 | 2 | 2 | 4 |
| | Sum | 17 | 16 | 14 | 13 | 13 | 73 |
| | | T1 subtype | | | | | |
| | | T1-Myc | T1-LumGU | T1-Inflam | T1-TLum | T1-Early | Sum |
| Lund subtype | Basal/ | 2 | 1 | 6 | 0 | 3 | 12 |
| | SCC-like | | | | | | |
| | Genomically | 3 | 14 | 0 | 0 | 0 | 17 |
| | unstable | | | | | | |
| | Mes-like | 0 | 0 | 5 | 0 | 0 | 5 |
| | Sc/NE-like | 0 | 0 | 0 | 0 | 1 | 1 |
| | Urothelial- | 12 | 1 | 3 | 13 | 9 | 38 |
| | like | | | | | | |
| | Sum | 17 | 16 | 14 | 13 | 13 | 79 |

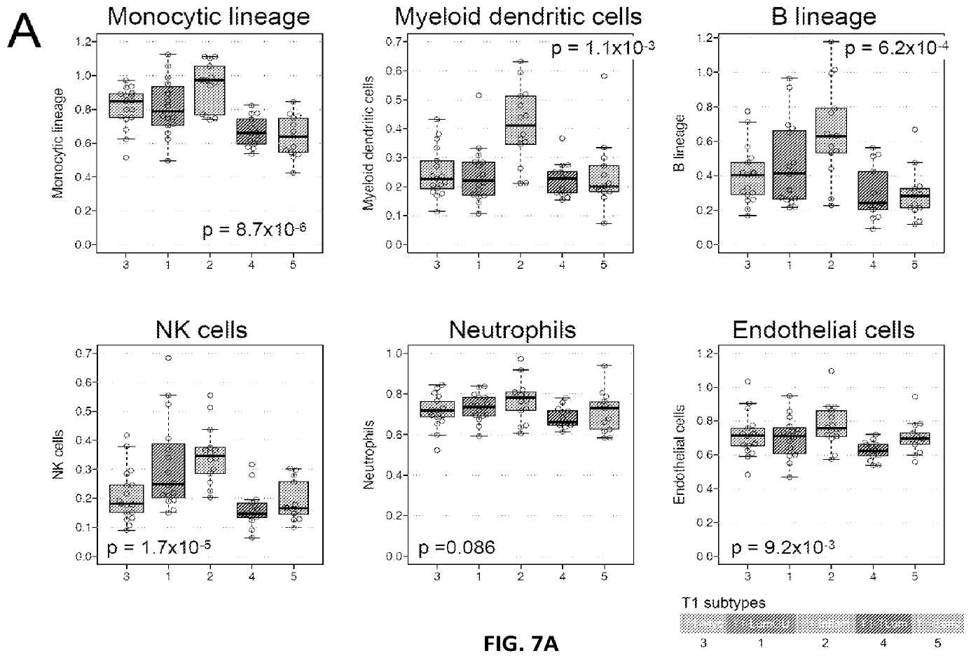

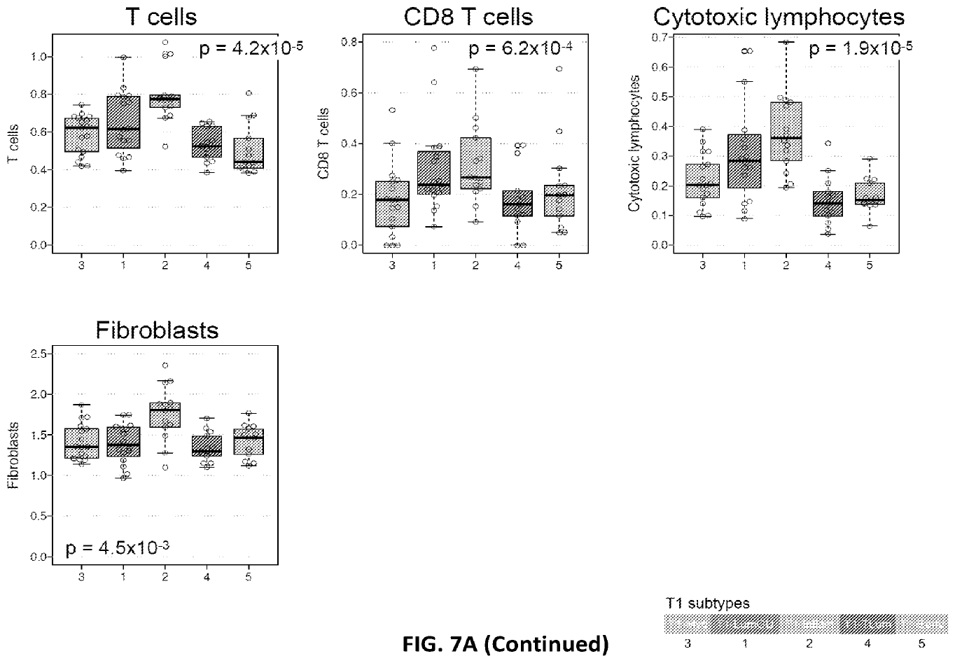

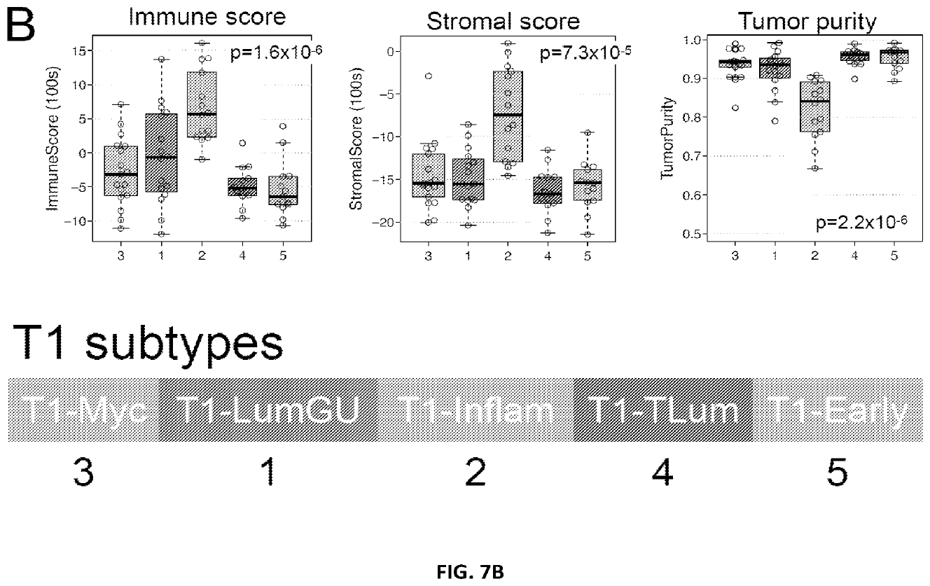

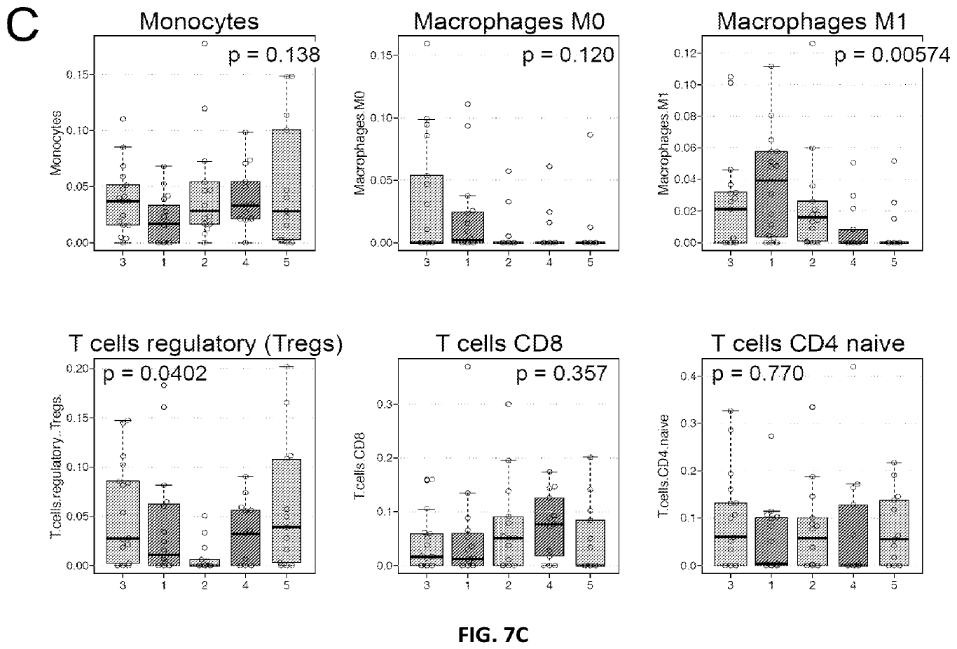

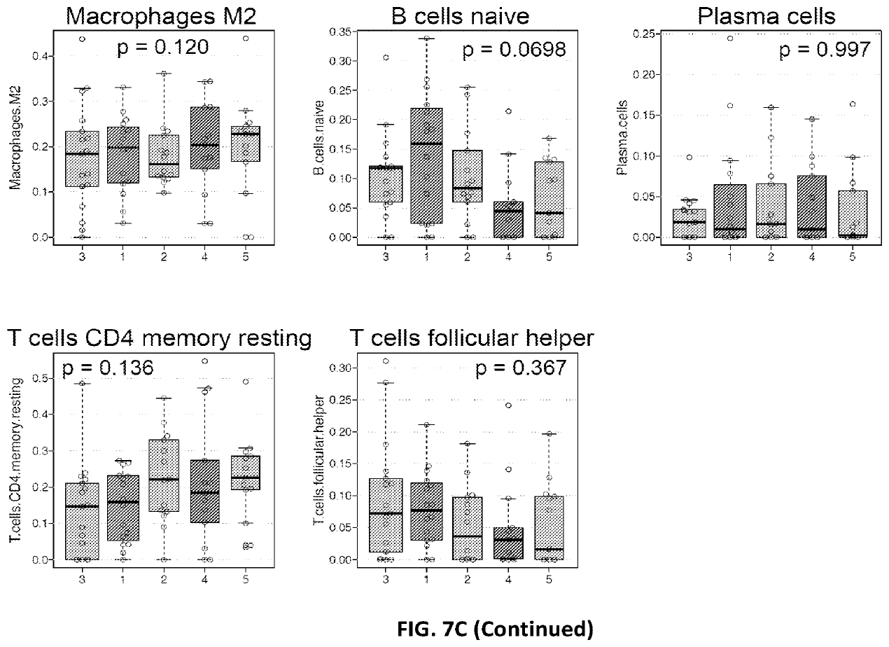

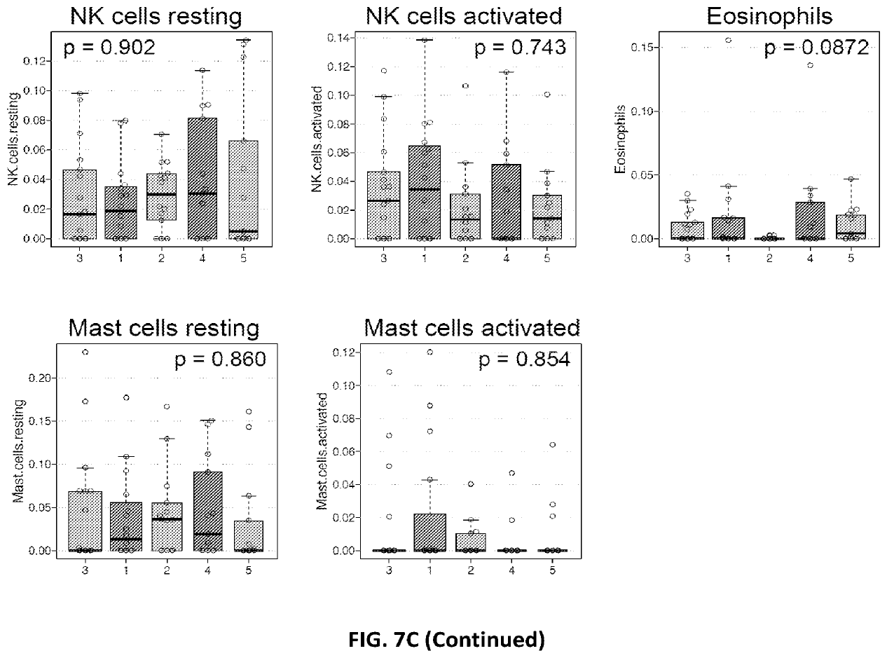

T1-Inflamed (S2). A set of 170 inflammation-related genes was highly expressed in T1-Inflamed (T1-Inflam) tumors (FIGS. 1A,D and 4C; AUC=0.91, CERNO test [5]). T1-Inflam's had the highest levels of many immune cell types, including cytotoxic lymphocytes and T cells, as well as the highest immune and stromal scores, and the lowest tumor purity (FIGS. 1F, 7B). Multiple inflammatory Hallmarks were enriched, and Myc and E2F target Hallmarks were repressed (FIG. 1C, 6). While T1-Inflams were mostly LumP (11/14, 79%), CIS rates were low (3/14, 21%) and there were 4/14 (29%) recurrences by 24 months. Hallmarks and immune signatures were consistent with increased expression of immune regulators NFATC2 and STAT4 (FIG. 5B), and we hypothesize that T1-Inflam tumors represent an immune-active and inflamed T1 subtype.

T1-Myc (S3). Collectively, subtypes T1-Myc and T1-Early (S5) and had the most recurrences, with over half of tumors experiencing a recurrence after BCG treatment (14/24, 58% of patients at 24 mo, over both subtypes, FIG. 1B). T1-Myc had the most recurrences over 24 months (12/17, 71%). T1-Myc tumors were mostly (14/17, 82%) LumP, and all were UROMOL class 2a. They had high MYC expression levels and enriched Myc target Hallmarks (AUC=0.71, FIGS. 1C, H, and 6). Inflammatory gene signatures were minimally repressed (AUC=0.66 FIG. 4C). Pathological CIS was present in 2/17 (12%), but CIS gene sets were unenriched (FIG. 4A).

T1-TLum (S4). Subtype T1-True Luminals (T1-TLum) was the most-luminal and urothelial-differentiated subtype. Overall, T1-TLums had the fewest 24-mo recurrences (2/13, 15%). T1-TLums had the highest median consensusMIBC LumP classifier score (FIG. 1E), and contained 4/13 (31%) UROMOL Class 1 tumors. T1-TLums had the fewest somatic CN gains [6] (FIG. 1G) and had strongly repressed CIS (i.e. enriched CIS DOWN with repressed CIS UP) (FIG. 4A). Inflammatory and proliferative Hallmarks were repressed (FIGS. 1C and 6), immune cell markers were low by multiple deconvolution methods (FIG. 7), and luminal differentiation genes FGFR3 and RXRA were highly expressed (FIG. 1H).

T1-Early (S5). Subtype T1-Early had 5/13 (38%) recurrences within 6 months of induction BCG, with no further recurrences by 24 months. This subtype had the highest median expression of MYC, and enriched Myc target Hallmarks (AUC=0.80, FIGS. 1C, H and 6). These tumors had no reported CIS, and had repressed CIS gene signatures (FIG. 4). While both T1-Myc and T1-Early had elevated MYC expression, T1-Early differed from T1-Myc in having repressed immune response Hallmarks for IFN-alpha (AUC=0.81) and IFN-gamma (AUC=0.75) (FIG. 6). Thus, T1-Early appeared to be a MYC-driven subtype with an immune-suppressive microenvironment that was depleted in immune cells, suggesting its tumor microenvironment may represent an immune desert. Grouping the two Myc-driven subtypes together, T1-Early and T1-Myc had significantly worse recurrence-free survival than the other three subtypes grouped (p=0.025, FIG. 1I).

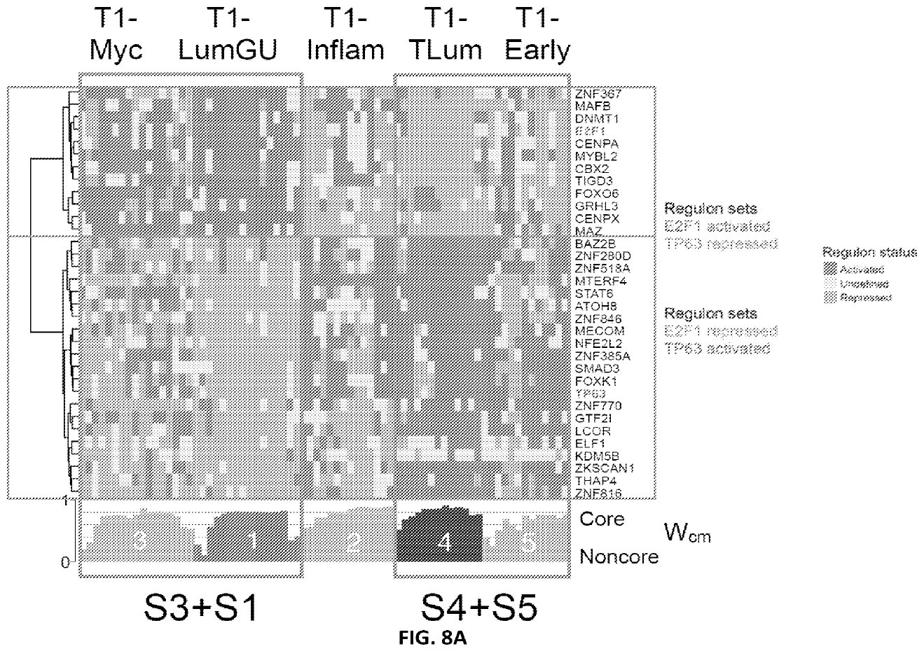

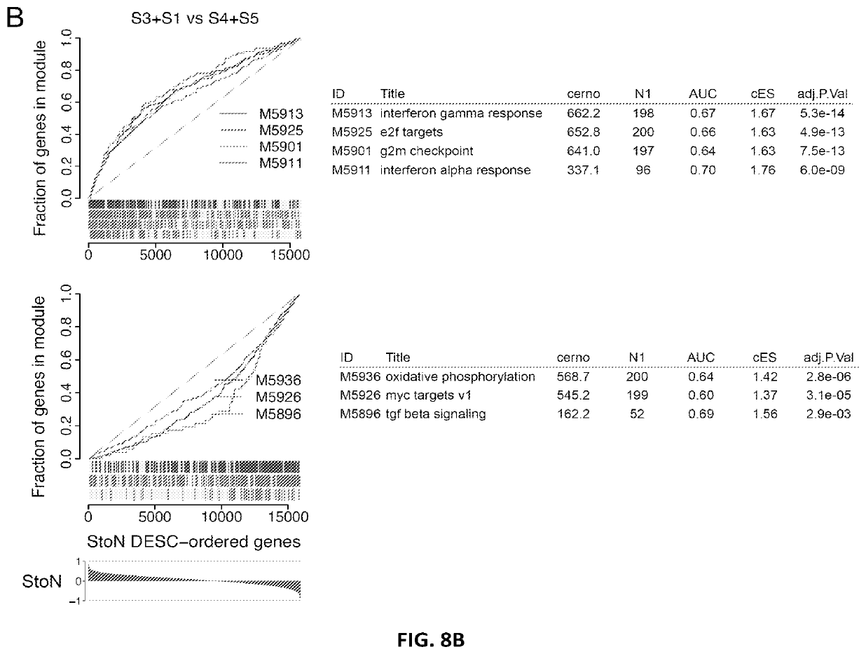

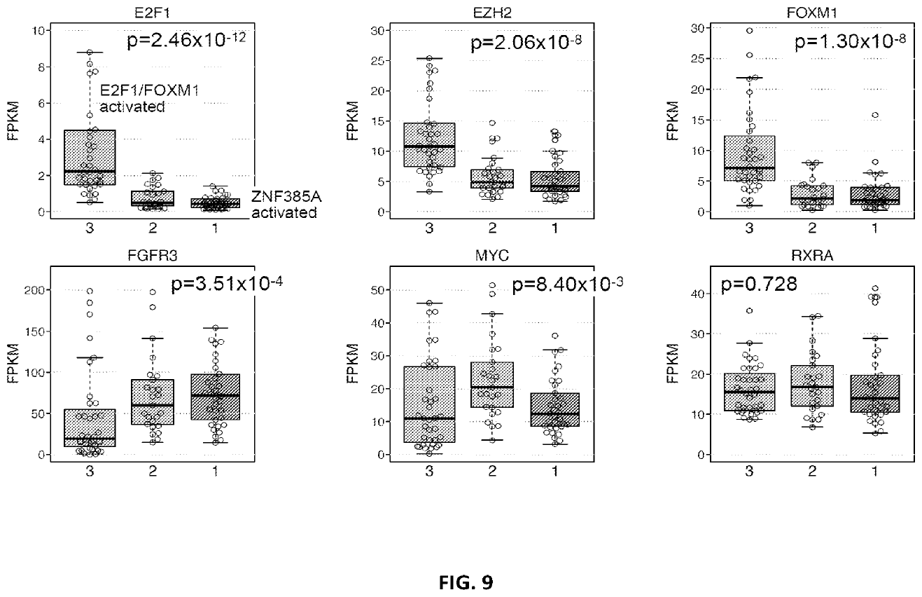

We used regulon analysis to further characterize the molecular differences and similarities of each subtype. This identified two major patterns of regulon activity that suggested that the five expression subtypes could be grouped into two regulon classes: T1-LumGU+T1-Myc and T1-TLum+T1-Early (FIG. 8A). Subtypes T1-LumGU+T1-Myc had activated regulons for transcription factors E2F1 and FOXM1, and enriched Hallmarks for E2F targets, G2M checkpoint, and interferon response pathways. In contrast, subtypes T1-TLum+T1-Early (and to some degree T1-Inflam) were characterized by activated SMAD3 and TP63 regulons, and enriched Hallmarks for TGF-Beta signaling, MYC targets, and oxidative phosphorylation (FIG. 1J, 8B). We found that these two major patterns regulon activity were also present in a cohort of 94 UROMOL T1 non-cystectomy tumors, where they were consistent with classifier subtyping, and with relative expression levels of E2F1 and other genes [7] (FIG. 9 and data not shown). That the five expression subtypes corresponded to two major regulon activity classes suggests that regulon activity profiles may provide coarser-grained groupings for T1 tumors.

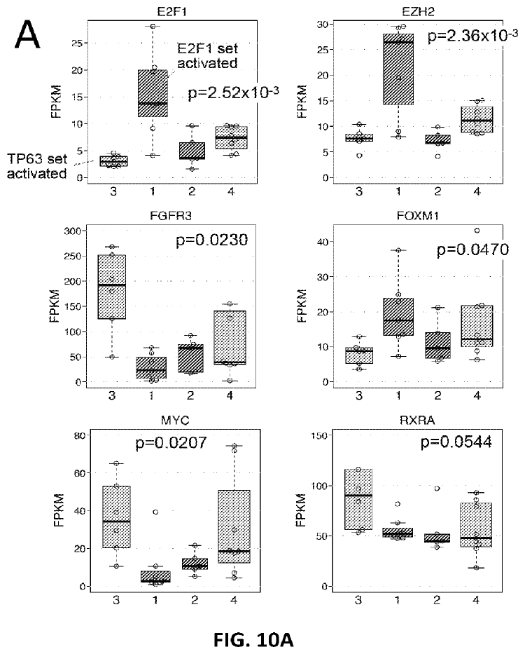

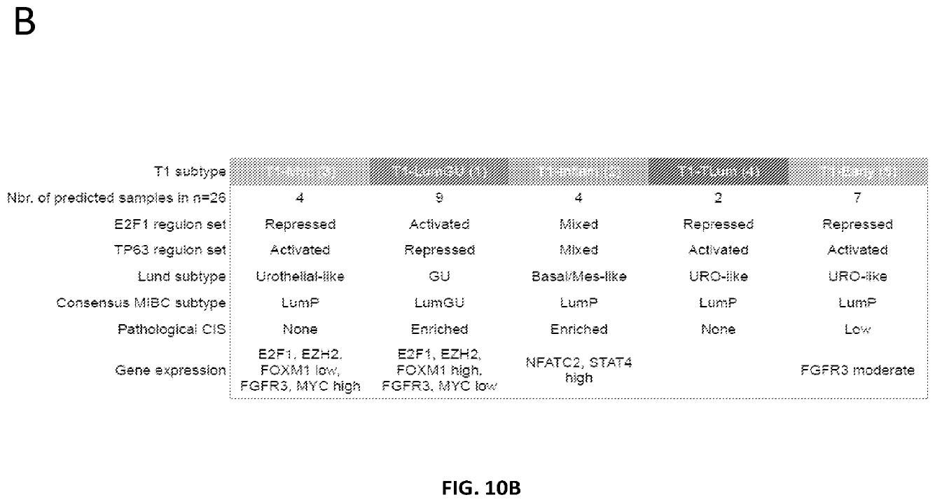

So that we could evaluate our subtypes in other patient cohorts, we developed an expression-based single-sample classifier for T1 tumors (https://github.com/csgroen/classifyT1BC). Since our discovery cohort was restricted to tumors treated with BCG, we evaluated tumors from a second cohort of 26 T1 patients from our institution, whose tumors had all been T1 on TUR, but who had elected to have an early cystectomy, rather than BCG treatment (Table 1B, FIG. 10 and data not shown). The classifier identified the five T1 subtypes in the 26 tumors, and, as validation of the classifier, we identified conserved regulon activity patterns, relative levels of gene expression, tumor subtypes, and pathologic CIS in the predicted subtypes (FIG. 10B). This result was consistent with the UROMOL results (FIG. 9 and data not shown) and showed that gene expression and regulons active within our five T1 subtypes were conserved in another cohort of T1 tumors, despite differences in outcomes, sample preparation and treatment.

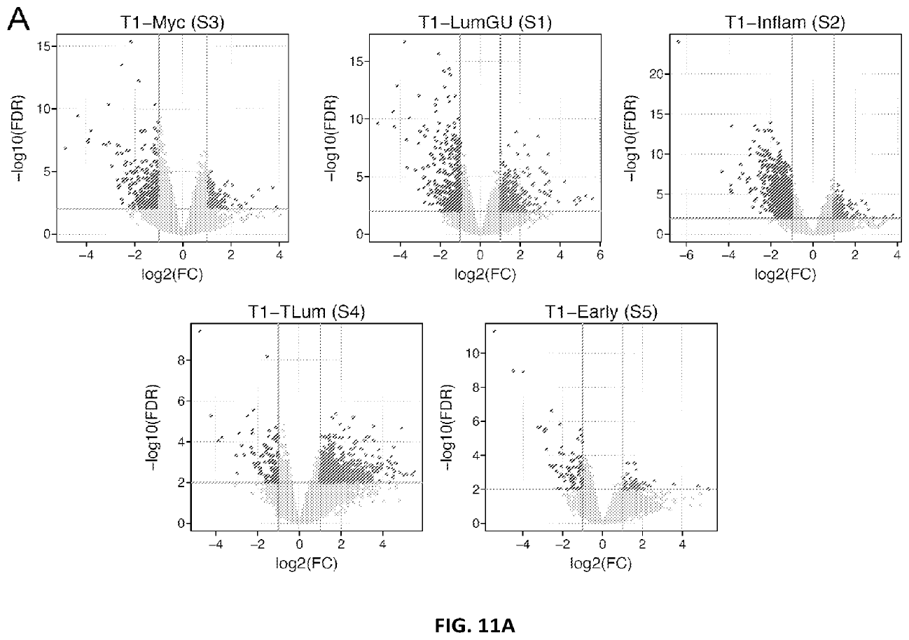

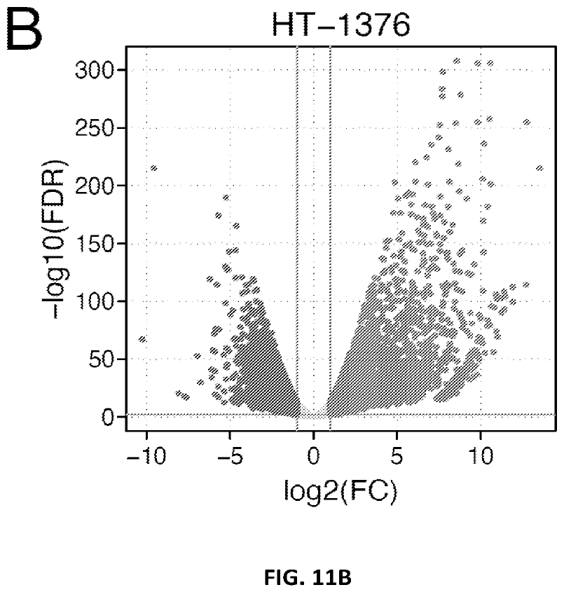

One potential application of T1 subtypes is to direct precision therapy targeting the unique features of the subtype. T1-LumGU tumors had the highest expression of E2F and its target EZH2. To test EZH2 as potential target in bladder cancer, we found that in vitro treatment of the luminal bladder cancer cell line HT-1376 with an EZH2 inhibitor strongly enhanced expression of DEGs that were repressed or underexpressed in T1-LumGU (FIGS. 11, 1K). While preliminary, these data suggest that consideration of the unique expression signature of each subtype, and/or its regulon network, may identify novel therapeutic targets of T1 tumors.

In summary, using a cohort of T1 patients treated with BCG, we identified five distinct molecular subtypes that appeared to be associated with two major classes of regulon activity, and we describe the characteristics of each subtype. In the future, evaluation of T1 subtypes may help risk-stratify T1 tumors and identify precision therapeutic targets.

### REFERENCES

[1] van den Bosch S, Alfred Witjes J. Long-term cancer-specific survival in patients with high-risk, non-muscle-invasive bladder cancer and tumour progression: a systematic review. European urology. 2011; 60:493-500.
[2] Meeks J J, Carneiro B A, Pai S G, Oberlin D T, Rademaker A, Fedorchak K, et al. Genomic characterization of high-risk non-muscle invasive bladder cancer. Oncotarget. 2016; 7:75176-84.
[3] Pennock N D, Jindal S, Horton W, Sun D, Narasimhan J, Carbone L, et al. RNA-seq from archival FFPE breast cancer samples: molecular pathway fidelity and novel discovery. BMC Med Genomics. 2019; 12:195.
[4] Kamoun A, de Reynies A, Allory Y, Sjodahl G, Robertson A G, Seiler R, et al. A Consensus Molecular Classification of Muscle-invasive Bladder Cancer. European urology. 2020; 77:420-33.
[5] Zyla J, Marczyk M, Domaszewska T, Kaufmann S H E, Polanska J, Weiner J. Gene set enrichment for reproducible science: comparison of CERNO and eight other algorithms. Bioinformatics. 2019; 35:5146-54.
[6] Beaubier N, Bontrager M, Huether R, Igartua C, Lau D, Tell R, et al. Integrated genomic profiling expands clinical options for patients with cancer. Nat Biotechnol. 2019; 37:1351-60.
[7] Hedegaard J, Lamy P, Nordentoft I, Algaba F, Hoyer S, Ulhoi B P, et al. Comprehensive Transcriptional Analysis of Early-Stage Urothelial Carcinoma. Cancer cell. 2016; 30:27-42.

### Example 3—Supplemental Methods for Example 2

Reference is made to “Supplemental Methods” for Robertson et al., “Identification of Differential Tumor Subtypes of T1 Bladder Cancer,” Eur. Urol., Volume 78, Issue 4, October 2020, Pages 533-537, the content of which is incorporated herein by reference in its entirety.

Patient cohorts, IRB, clinical data. IRB approval was obtained to evaluate a retrospective cohort of patients with stage T1 high grade bladder cancer at Northwestern Memorial Hospital; a waiver of informed consent was approved by Northwestern Institutional Review Board (STU00204352). All patients were newly diagnosed, with no prior bladder cancer, intravesical chemotherapy, or immunotherapy exposure. The date of TUR, reTUR and the amount of BCG received was recorded, with all patients completing at least induction (6 doses) of full-strength BCG. The clinical features of the cohort are listed in Table TA. Recurrence was defined as the development of any high-grade bladder tumor after the last TUR. Progression was considered any T2 or higher-grade lesion (including metastatic or nodal metastasis). A second cohort of n=27 patients with stage T1 high grade bladder cancer had received early cystectomy, rather than BCG treatment (Table 1B, see ‘Filtering’, below). The RNA of each cohort was extracted and sequenced separately.

Time to recurrence was determined as the time from the last TUR procedure to the diagnosis of pathologic recurrence.

FFPE RNA extraction, library construction, and sequencing. The paraffin block at diagnosis was used as the primary tissue for evaluation. Paraffin blocks were stored at room temperature. A GU pathologist evaluated every section and identified the high grade and invasive part of the tumor. This was then macrodissected for dual RNA and DNA extraction.

For the BCG-treated cohort (n=73), RNA was with the Roche HighPure miRNA Kit (Roche #05080576001), following the manufacturer's instructions. Stranded total RNA-seq was conducted in the Northwestern University NUSeq Core Facility. Briefly, RNA quantity was determined with a Qubit fluorometer. Total RNA examples were also checked for fragment sizing using Agilent Bioanalyzer 2100. The Illumina TruSeq Stranded Total RNA Library Preparation Kit was used to prepare sequencing libraries, following the manufacturer's procedure, which includes rRNA depletion with RiboZero Gold, cDNA synthesis, 3′ end adenylation, adapter ligation, library PCR amplification and validation. Libraries were pooled and sequenced on an lllumina HiSeq 4000, generating 50 bp single-end (SE) reads at a sequencing depth of 20-25M reads/sample.

For the early cystectomy cohort (n=27), RNA was extracted with the Qiagen Allprep DNA/RNA FFPE kit. Libraries were constructed with an Illumina TruSeq Stranded with RiboZero kit, and were sequenced as 150-bp PE reads on an Illumina HiSeq 4000, with a target depth of 20M read pairs per sample.

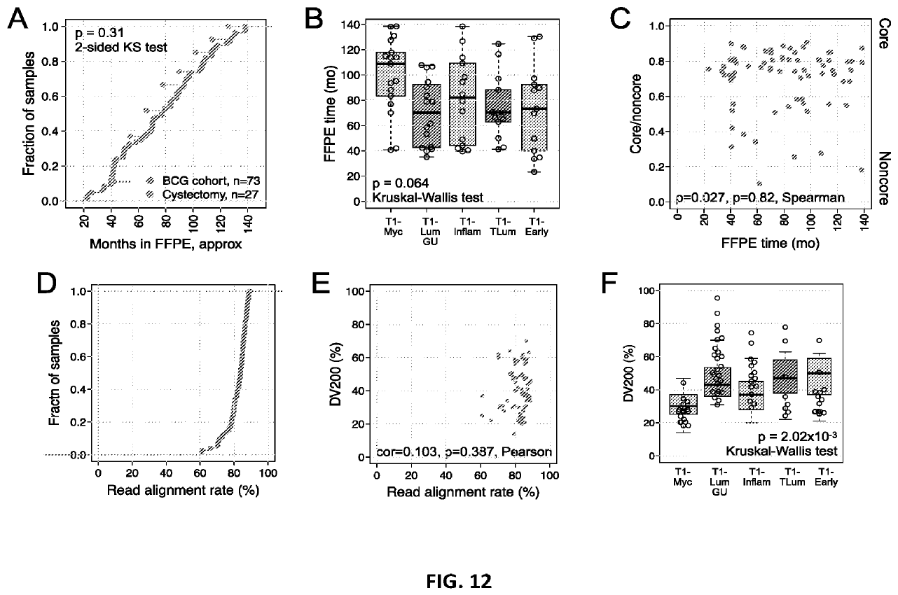

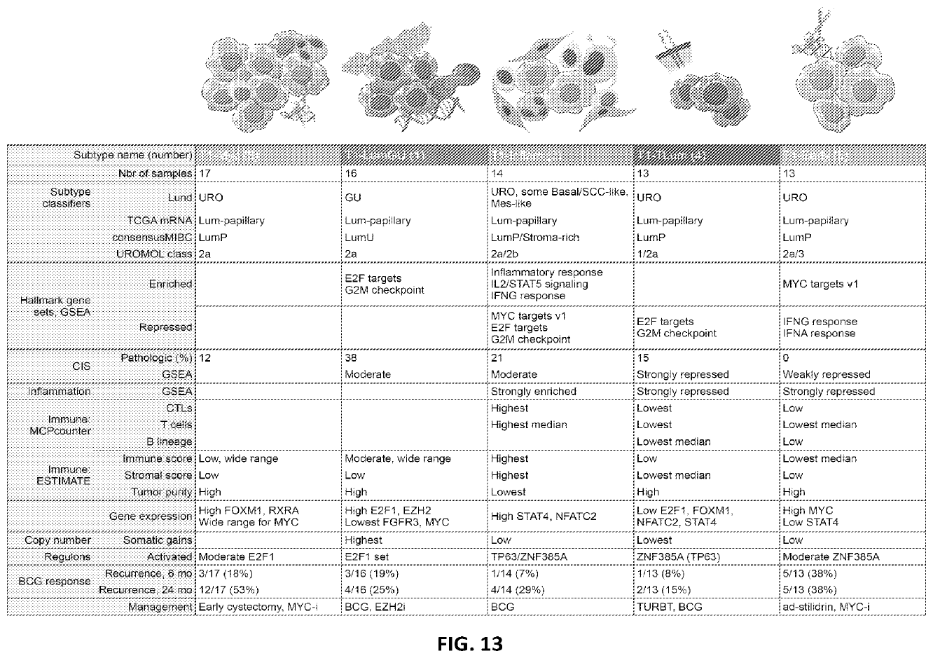

FFPE storage time. For the n=73 BCG cohort, surgery dates were given as day/month/year, and we assumed that RNA for all samples was extracted on 1 Jun. 2019. For the n=26 early cystectomy cohort, surgery dates were given as a year (e.g. 2016); we assumed that surgery dates were 1 June of the given year, and that RNA was extracted on 1 Dec. 2019. We compared the distributions of estimated FFPE storage times using a two-sided Kolmogorov-Smirnov test, in R v3.6.2. See FIG. 12.

Filtering RNA-seq data by the read alignment rate. For the BCG (n=73) and early cystectomy (n=27) cohorts, we generated an empirical distribution function of read alignment percentage, using R 3.6.2. For the cystectomy cohort, one sample had an atypically low alignment rate, and we removed it from the study by requiring at least 60% of reads to have aligned, leaving n=26 samples for analysis. All BCG-cohort samples satisfied this threshold, and we retained all n=73 of its samples (FIG. 12).

Quality metrics for RNA and RNA sequencing data. We compared distributions of estimated FFPE storage time for the two cohorts, using a two-sided Kolmogorov-Smirnov test, in R 3.6.2 (FIG. 12). We used a Kruskal-Wallis test to compare distributions of FFPE storage time across the five T1 subtypes. We assessed the relationship between FFPE storage time and the core/noncore cluster membership metric using R's cor.test function, with a Spearman correlation. We assessed the distribution of RNA-seq read alignment rates to the GRCh38 reference genome with an empirical distribution function. We assessed the relationship between the DV200 RNA quality metric and the read alignment rate with a Pearson correlation, and characterized differences in distributions of DV200 across the five T1 subtypes with a Kruskal-Wallis test.

Gene expression analysis. The n=73 cohort was sequenced with 50-bp SE reads. For the n=26 cohort, we approximated the n=73 cohort's sequence data by trimming the 150-bp reads to 50 bp with the FASTQ Toolkit v2.2.0, using default settings, and using only one read from each pair. For both cohorts we aligned the RNA sequence reads to the GRCh38.p12 reference genome sequence with STAR v2.6.0a, using settings: --outSAMtype BAM SortedByCoordinate --outFilterMismatchNmax 20 --outFilterType BySJout --outSJfilterCountUniqueMin -1 2 2 2 --outSJfilterCountTotalMin -1 2 2 2 --outFilterIntronMotifs RemoveNoncanonical --outReadsUnmapped Fastx --outFilterMultimapNmax 2.

To generate FPKM-normalized gene expression data, we processed BAM files with cuffquant from Cufflinks v2.2.1, using settings: -u -p 12 --library-type fr-firststrand, with an Ensembl v97 GRCh38 gene annotation GTF file, and then cuffnorm, using default settings.

To assess whether n=26 cohort results were sensitive to using the 150-bp PE RNA-seq data, rather than its 50-bp SE subset, we generated FPKMs using the full RNA-seq data set, using a similar approach.

To generate TPM-normalized gene expression data, so that we could compare FPKMs and TPMs (FIG. 5B), we processed RNA-seq read counts with DESeq2 v1.26.0, applying the following function (Wagner et al. 2012):

```
tpm <- function(counts, gene.length) {
  rpk <- counts / gen.length
  scale_factor <- sum(rpk) / 1e6
  rpk / scale_factor
}
```

Consensus subtypes from gene expression profiles. In the discovery cohort, we identified groups of samples that had similar expression profiles by unsupervised consensus clustering, applying ConsensusClusterPlus v1.50.0 (Wilkerson and Hayes, 2010) to log 10-transformed, mean-centered FPKM profiles for the 2945 protein-coding genes that had FPKM variances above that cohort's 85^{th} percentile. We considered hierarchical, PAM and k-means clustering, Pearson and Spearman distances, and solutions with between two and eight clusters. Our approach accepted that a cohort may have more than one reasonable clustering solution, and that, in such a case, choosing which solution is the most appropriate to report on in detail may depend on the questions being asked (Aine et al. 2015). Given this, we assessed consensus clustering membership heatmaps, dendrograms, core/noncore profiles (below), covariates, and Kaplan-Meier plots, and selected three clustering solutions for more detailed assessment: a) 3 clusters: Pearson, PAM, fitem=0.85, reps=20,000; b) 4 clusters: Pearson, k-means, fitem=0.875, reps=5000; and c) 5 clusters: Spearman, PAM, fitem=0.875, reps=50,000.

Cluster memberships of samples that were strongly non-core (i.e. atypical) cluster members tend to have lower technical repeatability (Lemer and Robertson 2016), so we identified core (i.e. more-typical) cluster members, vs noncore (i.e. more-atypical) cluster members by calculating a profile of silhouette widths from the consensus membership matrix (as in e.g. Robertson et al. 2017).

Genes that were differentially expressed in each T1 subtype. For the BCG-treated discovery cohort, we identified differentially expressed genes (DEGs) by applying edgeR v3.28.0 to read counts returned by HTSeq v0.11.2 for (GRCh38) Ensembl v97 gene annotations, retaining all gene biotypes. We filtered at CPM>10 and then did an edgeR quasi-likelihood F-test.

In separate calculations, we compared gene expression in each subtype to all other samples from the n=73 cohort. We generated volcano plots with a custom R script, requiring FDR<0.01 and |FC|>2.0, and retaining only protein-coding genes (FIG. 11 and data not shown).

For a DEG heatmap for the discovery cohort (FIG. 1A), we processed RNA-seq read counts with DESeq2 v1.26.0, using VST normalization, and filtered genes by subtype-specific |log FC|>2.0 and adjusted p<10⁻⁴. For the 508 protein-coding DEGs retained, we rescaled expression values to between −0.5 and 0.5, generated a heatmap with pheatmap v1.0.12, and labeled the heatmap's scalebar “Rescaled DESeq2 normalized counts”.

Gene set enrichment analysis for T1 subtypes. We used gene sets (i.e. modules) from MSigDB v7.0, and CERNO tests (typically using qval=0.05) with the tmod v0.40 package (Zyla et al. 2019) in R 3.6.2. We compared gene sets against reference gene lists that had a subtype-specific gene order, which we generated as follows. We extracted FPKMs for 15853 protein-coding genes by requiring that the gene's FPKM was at least 1.0 in at least 10 of 73 samples. For each subtype, we ordered the genes by the signal-to-noise (StoN) ratio for that subtype vs. samples in all other subtypes, calculating StoN as the difference of mean FPKMs in the two groups, divided by the sum of the standard deviations for the two groups. Analyses were done with the genes in descending (vs. ascending) StoN order, to identify gene sets that were enriched in genes that had relatively high (vs. low) StoN i.e. expression in a subtype. We did similar CERNO tests with 170 inflammation genes and CIS up/down genes (see below). Where gene sets were returned, we used tmod v0.40 to generate an evidencePlot, using the DESC gene order.

To summarize GSEA results, we used a custom R script to display coloured disks for 12 selected Hallmark gene sets, and for inflammation and CIS gene sets, following the example of tmod's panelPlots. Disks were red if the gene set was enriched in positive-StoN genes and blue if enriched in negative-StoN genes; we made the disk area to be proportional to the CERNO area under the curve (AUC), and the disk opacity proportional to −log 10(adjusted P value).

Gene set enrichment analysis for two regulon activity groups: S3+S1 vs. S4+S5. We noted two dominant regulon activity patterns in the discovery cohort. In T1-Myc and T1-LumGU (i.e. S3 and S1), a set of regulons that included E2F1 was activated and a set that included TP63 was repressed. In contrast, T1-TLum and T1-Early (i.e. S4 and S5), the E2F1 set was repressed and the TP63 set was activated. These results suggested that we compare Hallmarks for two groups of samples: group 1, consisting of S3+S1, and group 2, consisting of S4+S5. For FPKMs for 19630 Ensembl v97 coding genes that had unique gene symbols, we retained the 15853 for which FPKMs were >1.0 in >10 of the cohort's 73 tumor samples. Removing S2 samples left 59 tumour samples. We generated two-group signal-to-noise (StoN) values for the 15853 genes, where StoN was the difference of means divided by the sum of standard deviations. We then sorted the gene symbols by descending StoN. We ran CERNO tests with the tmod v0.40 package, with MSigCB v7.0 Hallmark gene sets. We removed gene sets with AUC<0.60, and selected a subset of gene sets that were relevant to questions we were addressing.

Gene sets for heatmaps and GSEA. 170 inflammation-related genes were from supplemental table 3 of (Robinson et al. 2019). Carcinoma in situ (CIS) genes were assessed in two sets that were from (Robertson et al. 2017). The first (‘up’) set was 31 genes whose expression should be higher in CIS: AKR1B10, CALD1, CDH11, CLIC4, COL15A1, COL3A1, CXCR4, DCN, DPYSL2, EFEMP1, FLNA, HLA-DQA1, HLA-DQB1, HOXA9, ITM2A, KPNA2, KYNU, LHFPL6, LUM, LYZ, MAN1C1, MSN, NR3C1, PDGFC, RARRES1, S100A8, SGCE, SPARC, TOP2A, TUBB, and UAP1. The second (‘down’) set was 36 genes whose expression should be lower in CIS: ACSBG1, ANXA10, BBC3, BCAM, BMP7, BST2, CA12, CLCA4, CRTAC1, CTSE, CYP2J2, EEF1A2, ENTPD3, FABP4, FGFR3, GRB7, HBG1, HOXA1, HOXB2, INA, ITGB4, IVL, KCNQ1, LAD1, LAMB3, LTBP3, MAPRE3, MST1R, PADI3, PLA2G2A, SOX15, TMPRSS4, TNNI2, TRIM29, UPK2, and UPK3B.

We generated heatmaps with pheatmap v1.0.12.

Regulon analysis. To assess regulons for the n=73 BCG cohort, we used RTN v2.11.6 to generate a transcriptional network from the coding gene expression data, setting RTN's 1612 transcription factors (TFs) from (Lambert et al. 2018) as the regulators. For the permutation step we used a pValueCutoff of 0.040, which we estimated with RTN's tni.alpha.adjust( ) function, taking as a reference a pValueCutoff of 10⁻⁶ for 300 tumour samples, and a beta value of 0.25. We applied 200 bootstraps to the initial network to generate the ‘reference’ transcriptional network, then generated a smaller, DPI-filtered network in which direct TF targets were enriched. We used RTN's tni.gsea2( ) function to calculate profiles across the n=73 cohort of regulon activity (dES), and of regulon activity status (activated, repressed, or undefined), requiring each TF regulon to have at least 15 positive and negative targets. We used pheatmap v1.0.12 with Euclidean distances and ward.D2 clustering to generate a heatmap of regulon status.

The early cystectomy cohort had n=26 samples, after we had removed one sample that had an atypically low read alignment rate (see above). To calculate regulon activities for this small cohort, we used RTN v2.11.6's tni.replace.samples( ) function, using the regulons from the n=73 cohort and the FPKM expression matrix from the n=26 cohort. As noted above, did this with FPKMs calculated from 50-bp SE reads, and then from the as-sequenced 150-bp PE reads. We applied RTN's 2-tailed GSEA calculation to the resulting ‘replaced’ ‘tni’ object, filtering by posANDneg=15, and extracted regulon status values {-1,0,1} for the regulons that passed the filter (these were the same regulons that had passed the filter for the n=73 cohort). We used pheatmap v1.0.12 to generate a heatmap, clustering rows and columns with a Euclidean distance and wardD2.

For an RTN run on the UROMOL n=94 non-cystectomy cohort, we used 20279 expressed coding genes that had unique gene symbols. We set a pValueCutoff=0.015, corresponding closely to alphaB returned from tni.alpha.adjust( ) for nB=94, nA=300, alphaA=10⁻⁶, and beta=0.25.

Consensus subtypes from regulon activity profiles. For the n=26 cohort, we generated a four-cluster solution by consensus clustering activity (dES) profiles for 33 regulons (see below), using ConsensusClusterPlus v1.50.0, Euclidean distances, k-means clustering, maxK=8, reps=20000, pItem=0.90, and pFeature=0.85.

For UROMOL n=94 cohort, we generated a 3-cluster solution from dES profiles using ConsensusClusterPlus v1.50.0, Pearson distances, k-means clustering, maxK=8, reps=50000, pItem=0.95, and pFeature=0.85.

Single-sample classifier. First, we performed ANOVA tests using log-transformed normalized (log₂ FPKM) RNA-seq gene expression data and the five class (i.e. subtype) calls from the n=73 discovery cohort to identify genes that were able to distinguish between the five classes. We adjusted the F-test p-values using the Benjamini-Hochberg method. We then filtered and ranked the genes by the F-test adjusted p-values and selected varying numbers of features (5, 10, 20, 30, 50, 100, 200, 300, 500, 700 and 1000) to fit an elastic net model. We used the caret R package (v. 6.0-86) to perform model testing using 5-fold cross-validation, and to tune the lamdba and alpha parameters. We selected the best model (300 features, α=0.1, λ=0), which achieved an average accuracy of 94.5% in the cross-validation. We used this single-sample classifier model to classify samples in the n=26 early cystectomy cohort. The classifier is available as an R package from https://github.com/csgroen/classifyT1BC.

Kaplan-Meier analyses. Analyses including calculating log-rank p values, were done with the R survival v3.1-8 package, in R 3.6.2.

Immune cell type deconvolution: MCPcounter. By requiring that a gene's FPKMs were >0.2 in at least 15 of 73 samples, we retained 17850 of 19632 coding genes, whose gene symbols made 99 of 111 MCPcounter markers available. We then processed log 10(FPKM+1) expression values with MCPcounter v1.1.0 (Becht et al. 2016). P values for the distribution of cell type results across consensus subtypes were calculated with Kruskal-Wallis Chi-square tests.

Immune cell type deconvolution: ESTIMATE. From the Ensembl v97 (GRCh38) FPKMs and GTF file, we extracted records 58735 genes, which included all biotypes. ESTIMATE v1.0.13 (Yoshihara et al. 2013) used 10412 ‘common_genes’, whose symbols were available with the ESTIMATE R package. From the Ensembl GTF records corresponding to common_genes, 12 gene symbols had two ENSGs each, and for these we retained only the record with the smaller ENSG. Our final set of FPKMs included 10014 (96%) of the ‘common_genes’. We applied a log 2 transformation to the FPKMs, to approximate microarray data. We wrote a GCT file for the log 2 FPKMs, then ran estimateScoreo with platform=“affymetrix”. We plotted boxwhisker plots with R 3.6.2, and assessed the distribution of scores and purity with a Kruskal-Wallis test.

Immune cell type deconvolution: CIBERSORT. We used the web server (https://cibersort.stanford.edu) (Newman et al. 2015), inputting an expression matrix for 17850 expressed coding genes, and setting all FPKMs that were <1×10⁻¹⁰ to zero. For the run we asked for 500 permutations and used the LM22 reference.

Treating cell lines with an EZH2 inhibitor, per-subtype GSEA for EZH2 and PRC2. HT-1376 (ATCC) cells were grown in RPMI medium, supplemented with Pen/Strep and 10% FBS. Cells were seeded in 6-well multi-well plates at a density of 200,000 cells per well, and grown for 7 days. Cells were passaged when confluent, and seeded as described before. Cells were treated with Epz0011989 (Epizyme, Cambridge Mass.) at a final concentration of 5 uM, or with DMSO (vehicle) for 7 days. Cells were harvested in Trizol, and RNA was extracted according to manufacturer's instructions. Biologic triplicates were done for both cell lines; each cell line was aliquoted in triplicate, treated and harvested and sequenced separately. For whole protein extracts, cells were lysed in-well by addition of 200 uL of whole cell extraction buffer (100 mM Tris-HCl, pH 8.0, 10% Glycerol, 2% SDS, 10 mM DTT, 1 mM PMSF).

Libraries were constructed using an Illurnina TruSeq stranded nRNA-seq library preparation kit. Libraries were sequenced with 50-sp SE reads on an Illumina HiSeq 4000, to a mean depth of 30 M reads per sample.

For the cell lines, RNA-seq analysis involved the following steps. Low-quality reads and adapters were removed with with Trimmomatic v0.33 using defaults. Trimmed reads were mapped to the GRCh37/hg19 genome with STAR v2.5.2 using settings: --chimSegmentMin 12 --quantMode TranscriptomeSAM --outReadsUnmapped Fastq --outMultimapperOrder Random --outSAMmapqUnique 60 --outFilterMultimapNmax 20 --outFilterMismatchNmax 2. Read alignments were sorted with Samtools v1.6. Reads were counted against Ensembl v75 GRCh37 gene annotations with HTSeq v0.11.2. Differentially expressed genes were identified with edgeR v3.12.1.

For GSEA, we ran a CERNO test (Zyla et al. 2019) on each subtype, for C2 and C6 gene sets involving EZH2 or PRC2, for descending and then ascending StoN, for that subtype, using a lenient qval=1.0 to allow weakly enriched or repressed gene sets to be returned.

We generated volcano plots with a custom R script, requiring adjusted p<0.01 and |FC|>2.0, and retaining only protein-coding genes.

Coding genes differentially up in EZH2i HT-1376 and down in T1-LumGU (S1). For that coding genes with FDR<0.01 and |FC|>2.0 that we'd used for volcano plots, we intersected the two lists of gene symbols to identify genes-of-interest. We regenerated the T1-LumGU volcano plot, colouring the genes-of-interest orange (FIG. 1K). Then, for these genes-of-interest, we used pheatmap v1.0.12 to generate a heatmap of FPKMs for the triplicate DMSO controls and the triplicate EZH2i treatments (data not shown).

UROMOL classes. UROMOL subtypes are available as 3 classes from (Hedegaard et al. 2016). Four-class subtype calls were supplied for the UROMOL cohort and were generated for the n=73 discovery cohort (L. Dyrskjot, personal communication).

DNA extraction, library construction, and exome sequencing. After RNA had been isolated (see above), pellets containing DNA and protein were extracted further using and AllPrepFFPE Kit (Qiagen #80234). Genomic DNA (gDNA) was quantified by Qubit and assessed for quality on an Agilent Bioanalyzer. We used the Illumina TruSeq DNA Exome Kit for all steps of library construction. Briefly, the gDNA was fragmented to 150 bp insert size using Covaris shearing, which was followed by end repair, library size selection, and 3′ end adenylation. Multiple-index adapters were then ligated to the ends of the DNA fragments. A limited-cycle-number PCR was used to selectively enrich DNA fragments that had adapters ligated on both ends. After being validated with Qubit and Agilent Bioanalyzer, the DNA libraries carrying unique barcoding indexes were pooled and hybridized to exome oligo probes to capture the exonic regions of the genome. The 45 Mb capture probes target ≥98% of RefSeq, CCDS, and Ensembl coding exons (https://www.illumina.com/products/by-type/sequencing-kits/library-prep-kits/truseq-exome.html). The capture process was conducted twice to ensure high specificity for captured regions. After cleanup, the captured libraries were amplified with an 8-cycle PCR. After a post-PCR purification step, the enriched libraries were validated with Qubit quantification and a Bioanalyzer quality check using a High Sensitivity DNA chip. Libraries were sequenced on an Illumina HiSeq 4000 as PE 75 bp reads with dual indexing.

Copy number alterations (CNA). Somatic copy number alterations were estimated on the GRCh37/hgl9 reference genome by Tempus Labs (Chicago Ill.) (Beaubier 2019). Using hgl9 chromosome lengths from the UCSC genome browser, we calculated the fraction of the genome length that the CNA data indicated had CN gains. We displayed this across consensus subtype as a box-whisker plot, and calculated a p value using a Kruskal-Wallis test, all in base R 3.6.2.

### REFERENCES

Aine M, Eriksson P, Liedberg F, Sjodahl G, Hoglund M. Biological determinants of bladder cancer gene expression subtypes. Sci Rep. 2015 Jun. 8; 5:10957. doi: 10.1038/srep10957. PMID: 26051783.
Beaubier N, Bontrager M, Huether R, Igartua C, Lau D, Tell R, Bobe A M, Bush S, Chang A L, Hoskinson D C, Khan A A, Kudalkar E, Leibowitz B D, Lozachmeur A, Michuda J, Parsons J, Perera J F, Salahudeen A, Shah K P, Taxter T, Zhu W, White K P. Integrated genomic profiling expands clinical options for patients with cancer. Nat Biotechnol. 2019 November; 37(11):1351-1360. doi: 10.1038/s41587-019-0259-z. PMID: 31570899.
Becht E, Giraldo N A, Lacroix L, Buttard B, Elarouci N, Petitprez F, Selves J, Laurent-Puig P, Sautès-Fridman C, Fridman W H, de Reyniès A. Estimating the population abundance of tissue-infiltrating immune and stromal cell populations using gene expression. Genome Biol. 2016 Oct. 20; 17(1):218. PMID: 27765066.
Hedegaard J, Lamy P, Nordentoft I, et al. Comprehensive Transcriptional Analysis of Early-Stage Urothelial Carcinoma. Cancer Cell. 2016; 30(1):27-42. doi:10.1016/j.ccell.2016.05.004.
Lambert S A, Jolma A, Campitelli L F, Das P K, Yin Y, Albu M, Chen X, Taipale J, Hughes T R, Weirauch M T. The Human Transcription Factors. Cell. 2018 Feb. 8; 172(4):650-665. doi: 10.1016/j.cell.2018.01.029. PMID: 29425488.
Lerner S P, Robertson A G. Molecular Subtypes of Non-muscle Invasive Bladder Cancer. Cancer Cell. 2016 Jul. 11; 30(1):1-3. doi: 10.1016/j.ccell.2016.06.012. PMID: 27411578.
Liberzon A, Birger C, Thorvaldsdóttir H, Ghandi M, Mesirov J P, Tamayo P. The Molecular Signatures Database (MSigDB) hallmark gene set collection. Cell Syst. 2015 Dec. 23; 1(6):417-425. PMID: 26771021.
Newman A M, Liu C L, Green M R, Gentles A J, Feng W, Xu Y, Hoang C D, Diehn M, Alizadeh A A. Robust enumeration of cell subsets from tissue expression profiles. Nat Methods. 2015 May; 12(5):453-7. doi: 10.1038/nmeth.3337. PMID: 25822800.
Robertson A G, Kim J, Al-Ahmadie H, Bellmunt J, Guo G, Cherniack A D, Hinoue T, Laird P W, Hoadley K A, Akbani R, Castro M A A, Gibb E A, Kanchi R S, Gordenin D A, Shukla S A, Sanchez-Vega F, Hansel D E, Czerniak B A, Reuter V E, Su X, de Sa Carvalho B, Chagas V S, Mungall K L, Sadeghi S, Pedamallu C S, Lu Y, Klimczak L J, Zhang J, Choo C, Ojesina A I, Bullman S, Leraas K M, Lichtenberg T M, Wu C J, Schultz N, Getz G, Meyerson M, Mills G B, McConkey D J; TCGA Research Network, Weinstein J N, Kwiatkowski D J, Lerner S P. Comprehensive Molecular Characterization of Muscle-Invasive Bladder Cancer. Cell. 2017 Oct. 19; 171(3):540-556.e25. doi: 10.1016/j.cell.2017.09.007. PMID: 28988769.
Robinson B D, Vlachostergios P J, Bhinder B, Liu W, Li K, Moss T J, Bareja R, Park K, Tavassoli P, Cyrta J, Tagawa S T, Nanus D M, Beltran H, Molina A M, Khani F, Miguel Mosquera J, Xylinas E, Shariat S F, Scherr D S, Rubin M A, Lerner S P, Matin S F, Elemento O, Faltas B M. Upper tract urothelial carcinoma has a luminal-papillary T-cell depleted contexture and activated FGFR3 signaling. Nat Commun. 2019; 10(1):2977. doi:10.1038/s41467-019-10873-v. PMID: 31278255.
Wagner G P, Kin K, Lynch V J. Measurement of mRNA abundance using RNA-seq data: RPKM measure is inconsistent among samples. Theory Biosci. 2012; 131(4):281-285. doi:10.1007/s12064-012-0162-3. PMID: 22872506.
Wilkerson M D, Hayes D N. ConsensusClusterPlus: a class discovery tool with confidence assessments and item tracking. Bioinformatics. 2010 Jun. 15; 26(12):1572-3. doi: 10.1093/bioinformatics/btq170. PMID: 20427518.
Yoshihara K, Shahmoradgoli M, Martinez E, Vegesna R, Kim H, Torres-Garcia W, Treviño V, Shen H, Laird P W, Levine D A, Carter S L, Getz G, Stemke-Hale K, Mills G B, Verhaak R G. Inferring tumour purity and stromal and immune cell admixture from expression data. Nat Commun. 2013; 4:2612. doi: 10.1038/ncomms3612. PMID: 24113773.
Zyla J, Marczyk M, Domaszewska T, Kaufmann S H E, Polanska J, Weiner J. Gene set enrichment for reproducible science: comparison of CERNO and eight other algorithms. Bioinformatics. 2019 Dec. 15; 35(24):5146-5154. doi: 10.1093/bioinformatics/btz447. PMID: 31165139.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A method for classifying a sample of Stage T1 bladder cancer from a subject, the method comprising performing one or more of the following detecting steps:
(a) detecting activation and/or repression in the sample of one or more regulons selected from E2F1, TP63, and ZNF385A;
(b) detecting an increase in somatic copy number;
(c) detecting expression in the sample of one or more genes selected from FOXM1, RXRA, MYC, E2F1, EZH2, FGFR3, STAT4, and NFATC2;
(d) detecting tumor purity for the sample;
(e) detecting an immune score and/or a stromal score for the sample;
(f) detecting immune cells in the sample;
(g) detecting inflammation in the sample;
(h) detecting carcinoma in situ (CIS) in the sample, and/or detecting expression and/or repression of genes associated with CIS in the sample;
(i) detecting expression of one or more genes selected from E2F target genes, G2M checkpoints genes, inflammatory response genes, IL2/STAT5 signaling genes, IFNG response genes, INFA response genes, and MYC target genes;
and optionally performing the following classifying step:
(j) classifying the sample by one or more subtype classifiers selected from Lund, TCGA mRNA subtype classifier, consensusMIBC, and UROMOL class;
thereby classifying the Stage T1 bladder cancer.

2. The method of claim 1 comprising performing two or more of steps (a)-(j).

3. The method of claim 1 comprising performing three or more of steps (a)-(j).

4. The method of claim 1 comprising performing four or more of steps (a)-(j).

5. The method of claim 1 comprising performing five or more of steps (a)-(j).

6. The method of claim 1 comprising performing six or more of steps (a)-(j).

7. The method of claim 1 comprising performing seven or more of steps (a)-(j).

8. The method of claim 1 comprising performing eight or more of steps (a)-(j).

9. The method of claim 1 comprising performing nine or more of steps (a)-(j).

10. The method of claim 1 comprising performing all ten steps (a)-(j).

11. The method of claim 1, comprising: classifying the Stage TS1 bladder cancer into one of five (5) classes as follows: class 1 (which optionally may be referred to as “T1-LumGU”); class 2 (which optionally may be referred to as “T1-Inflam”), class 3 (which optionally may be referred to as “T1-Myc”), class 4 (which optionally may be referred to as “T1-TLum”), and class 5 (which optionally may be referred to as “T1-Early”), wherein:
class 1 (which optionally may be referred to as “T1-LumGU”), is defined by one or more of the following criteria:
(a) activation of the E2F1 regulon relative to class 2, class 3, class 4, and/or class 5;
(b) a high increase in somatic copy number relative to class 2, class 4, and/or class 5;
(c) relatively high expression of E2F1 and EZH2 in comparison to class 2, class 3, class 4, and/or class 5; and relatively low expression of FGFR2 and MYC in comparison to class 2, class 3, class 4, and/or class 5;
(d) high tumor purity;
(e) a moderate immune score and/or a low stromal score;
(h) presence of CIS, and/or increased or decreased expression of genes associated with CIS;
(i) enriched expression of E2F target genes and/or G2M checkpoints genes relative to class 2, class 3, class 4, and/or class 5; and
(j) a classification selected from Lund:GU, TCGA mRNA:Lum-papillary, consensus MIBC:LumU, and UROMOL class:2a;
class 2 (which optionally may be referred to as “T1-Inflam”), is defined by one or more of the following criteria:
(a) activation of the TP63/ZNF385A regulon relative to class 1, class 3, and/or class 5;
(b) a low increase in somatic copy number relative to class 1;
(c) relatively high expression of STAT4 and NFATC2 in comparison to class 1, class 3, class 4, and/or class 5;
(d) low tumor purity relative to class 1, class 3, class 4, and/or class 5;
(e) a high immune score and/or a high stromal score relative to class 1, class 3, class 4, and/or class 5;
(f) detected immune cells;
(g) detected inflammation;
(h) presence of CIS, and/or increased or decreased expression of genes associated with CIS;
(i) enrichment in expression of inflammatory response genes, IL2/STAT5 signaling genes, and IFNG response genes, and MYC target genes; and repression of expression of E2F target genes, G2M checkpoint genes, and MYC target genes relative to class 1, class 3, and/or class 5; and
(j) a classification selected from Lund:(URO, some Basal/SCC-like, Mes-like), TCGA mRNA:Lum-papillary, consensusMIBC:LumP/Stroma-rich, and UROMOL class:2a/2b;
class 3 (which optionally may be referred to as “T1-Myc”), is defined by one or more of the following criteria:
(a) moderate activation of the E2F1 regulon relative to class 1, class 2, class 4, and/or class 5;
(c) relatively high expression of FOXM1 and RXRA in comparison to class 1, class 2, class 4, or class 5, and a wide range of expression for MYC in comparison to class 1, class 2, and/or class 4;
(d) high tumor purity;
(e) a low immune score and/or a low stromal score;
(h) presence of CIS, and/or increased or decreased expression of genes associated with CIS; and
(j) a classification selected from Lund:URO, TCGA mRNA:Lum-papillary, consensusMIBC:LumP, and UROMOL class:2a;
class 4 (which optionally may be referred to as “T1-TLum”), is defined by one or more of the following criteria:
(a) activation of the ZNF385A (TP63) regulon relative to class 1, class 2, and/or class 5;
(b) a low increase in somatic copy number relative to class 1, class 2, and/or class 5;
(c) relatively low expression of E2F1, FOXM1, STAT4, and NFATC2 in comparison to class 1, class 2, class 3, and/or class 5;
(d) high tumor purity;
(e) a low immune score and/or a low stromal score;
(h) presence of CIS, and/or increased or decreased expression of genes associated with CIS;
(i) repression of expression E2F target genes and G2M checkpoint genes relative to class 1, class 3, and/or class 5; and
(j) a classification selected from Lund:URO, TCGA mRNA:Lum-papillary, consensusMIBC:LumP, and UROMOL class:1/2a;
class 5 (which optionally may be referred to as “T1-Early”), is defined by one or more of the following criteria:
(a) moderate activation of the ZNF385A regulon relative to class 1, class 2, class 3, and/or class 4;
(b) a low increase in somatic copy number relative to class 1;
(c) relatively high expression of MYC in comparison to class 1, class 2, or class 4;
(d) high tumor purity;
(e) a low immune score and/or a low stromal score;
(h) absence of CIS, and/or increased or decreased expression of genes associated with CIS in the sample;
(i) enrichment in expression of MYC target genes; and repression of expression of IFNG genes and IFNA genes relative to class 1, class 2, and class 4; and
(j) a classification selected from Lund:URO, TCGA mRNA:Lum-papillary, consensusMIBC:LumP, and UROMOL class:2a/3.

12-16. (canceled)

17. The method of claim 1, wherein detecting expression comprises detecting mRNA of a gene in the sample.

18. The method of claim 11, further comprising administering therapy for bladder cancer to the subject after classifying the sample of Stage 1 bladder cancer.

19. The method of claim 18, wherein the sample is classified as class 1 (which optionally may be referred to as “T1-LumGU”), and administering therapy to the subject comprises administering BCG therapy and/or EZH2-i therapy to the subject.

20. The method of claim 18, wherein the sample is classified as class 2 (which optionally may be referred to as “T1-Inflam”), and administering therapy to the subject comprises administering BCG therapy to the subject.

21. The method of claim 18, wherein the sample is classified as class 3 (which optionally may be referred to as “T1-Myc”), and administering therapy to the subject comprises performing a cystectomy on the subject and/or administering Myc-I therapy to the subject.

22. The method of claim 18, wherein the sample is classified as class 4 (which optionally may be referred to as “T1-TLum”), and administering therapy to the subject comprises administering TURBT or BCG to the subject.

23. The method of claim 18, wherein the sample is classified as class 5 (which optionally may be referred to as “T1-Early”), and administering therapy to the subject comprises administering ad-stilidrin and/or MYC-i therapy to the subject.

24. A system for classifying a sample of Stage 1 bladder cancer from a subject, the system comprising a computer processor configured for receiving input obtained by performing one or more of the following detecting steps:
(a) detecting activation and/or repression in the sample of one or more regulons selected from E2F1, TP63, and ZNF385A;
(b) detecting an increase in somatic copy number;
(c) detecting expression in the sample of one or more genes selected from FOXM1, RXRA, MYC, E2F1, EZH2, FGFR3, STAT4, and NFATC2;
(d) detecting tumor purity for the sample;
(e) detecting an immune score and/or a stromal score for the sample;
(f) detecting immune cells in the sample;
(g) detecting inflammation in the sample;
(h) detecting carcinoma in situ (CIS) in the sample, and/or detecting expression and/or repression of genes associated with CIS in the sample;
(i) detecting expression of one or more genes selected from E2F target genes, G2M checkpoints genes, inflammatory response genes, IL2/STAT5 signaling genes, IFNG response genes, INFA response genes, and MYC target genes; and
optionally performing the following classifying step:
(j) classifying the sample by one or more subtype classifiers selected from Lund, TCGA mRNA subtype classifier, consensusMIBC, and UROMOL class;
wherein the computer processor provides an output comprising a classification for the sample of Stage 1 bladder cancer based on the input.
